(19) United States
(12) Patent Application Publication
(10) Pub. No.: US 2020/0222251 A1
(43) Pub. Date: Jul. 16, 2020

(54) BELTED STRUCTURE WITH TACKIFIER-FREE ADHESIVE

(71) Applicant: The Procter & Gamble Company, Cincinnati, OH (US)

(72) Inventor: Robert Haines TURNER, Cincinnati, OH (US)

(21) Appl. No.: 16/830,446

(22) Filed: Mar. 26, 2020

Related U.S. Application Data
(63) Continuation of application No. 15/377,061, filed on Dec. 13, 2016.
(60) Provisional application No. 62/267,548, filed on Dec. 15, 2015.

(51) Int. Cl. A61F 13/49; C09J 123/20; A61F 13/56; A61F 13/64; C09J 123/14; A61F 13/515; C09J 121/00; C08L 23/22; C08L 23/20; A61F 13/496; A61F 13/539; A61L 15/24; A61L 15/58; A61L 15/60

(52) U.S. Cl. CPC A61F 13/49058; A61F 2013/53908; A61F 13/565; A61F 13/64; C09J 123/14; A61F 13/515; C09J 121/00; C08L 23/22; C08L 23/20; A61F 13/49011; A61F 13/496; A61F 13/539; A61L 15/24; A61L 15/585; A61L 15/60; C08L 2207/02; A61F 2013/49038; C09J 123/20

(57) ABSTRACT

Pull-on disposable absorbent articles with elastomeric belts are provided. The elastomeric belts include an outer nonwoven, an inner nonwoven, and elastic strands disposed therebetween. The elastomeric belts further have a front belt portion having a front waist edge, and front left and right side edges, and a rear belt portion having a rear waist edge and rear left and right side edges. An adhesive bonds at least one of the group consisting of the belt structure to the central chassis; the belt structure outer nonwoven to the belt structure inner nonwoven; the elastic strands to the belt structure nonwovens; and the respective front and rear left side edges and the front and rear right side edges. The adhesive is substantially tackifier-free.

### CROSS REFERENCE TO RELATED APPLICATIONS

This application claims priority, under 35 U.S.C. § 120, to U.S. application Ser. No. 15/377,061, filed on Dec. 13, 2016, which claims the benefit, under 35 U.S.C. § 119(e), of U.S. Provisional Application Ser. No. 62/267,548, filed on Dec. 15, 2015, both of which are incorporated by reference herein.

### FIELD

The present disclosure generally relates to pull-on disposable absorbent articles comprising an elastomeric belt and central chassis with tackifier-free adhesive bonds.

### BACKGROUND

A particular type of absorbent article pant design currently marketed is sometimes called the “balloon” pant. The balloon pant design usually includes a central absorbent chassis and an elastic belt. The elastic belt is usually relatively wide (in the longitudinal direction) and elastically stretchable in the lateral direction. It entirely encircles the wearer's waist, and thereby covers a relatively large amount of the wearer's skin, and also makes up a relatively large portion of the visible outside surfaces of the pant. The central chassis portion is typically joined to the inside of the belt in the front, wraps under the wearer's lower torso between the legs, and is joined to the inside of the belt in the rear. As such, balloon pants are a compilation of separate article components. Thus, it can be challenging to create an absorbent article wherein the materials that are bonded together form strong and stable bonds.

Molten adhesives used in assembling articles are typically made by combining polymer with additive components in a substantially uniform thermoplastic blend. However, the additive components, such as tackifiers, for example, can migrate during product use and create instability issues that negatively affect the performance and consumer impression of the article. In addition, for some hot melt adhesives, tackifiers may be a significant portion of the overall formulation and/or the most expensive component in the hot melt adhesive. Therefore, there is a continuing need to minimize the cost and minimize stability issues that adhesive with tackifiers may have.

Accordingly, there is a need for adhesives used in assembling balloon pants that have reduced amounts of tackifier or that are substantially free of tackifiers.

### BRIEF DESCRIPTION OF THE DRAWINGS

FIG. 1 is a perspective view of one example of a belted article.

FIG. 2 is a schematic plan view of a belted article precursor structure, prior to joining of the front and rear sections of the belt.

FIGS. 3A-3C are varying longitudinal cross-section views taken at line 3-3 of FIG. 2.

FIG. 4 is a longitudinal cross-section view taken at line 4-4 of FIG. 2.

### DETAILED DESCRIPTION

Various non-limiting embodiments of the present disclosure will now be described to provide an overall understanding of the principles of the structure, function, manufacture, and use of the absorbent articles disclosed herein. One or more examples of these non-limiting embodiments are illustrated in the accompanying drawings. Those of ordinary skill in the art will understand that the absorbent articles described herein and illustrated in the accompanying drawings are non-limiting example embodiments and that the scope of the various non-limiting embodiments of the present disclosure are defined solely by the claims. The features illustrated or described in connection with one non-limiting embodiment may be combined with the features of other non-limiting embodiments. Such modifications and variations are intended to be included within the scope of the present disclosure.

The following term explanations may be useful in understanding the present disclosure:

“Absorbent article” refers to pull-on garments generally worn by infants and other incontinent individuals to absorb and contain urine, feces and/or menses. It should be understood, however, that the term absorbent article is also applicable to other garments such as training pants, incontinent briefs, feminine hygiene garments or panties, and the like. In some embodiments, “absorbent article” may refer to a taped diaper.

The terms “elastic,” “elastomer,” and “elastomeric” refer to a material which generally is able to extend to a strain of at least 50% without breaking or rupturing, and is able to recover substantially to its original dimensions after the deforming force has been removed.

As used herein, “graphic” refers to formation of an object, which may or may not be colored. A graphic, however, does not include a field of color alone, wherein no formation of an object exists.

“Lateral”, with respect to a pant and its wearer, refers to the direction generally perpendicular with the wearer's standing height, or the horizontal direction when the wearer is standing. “Lateral” is also the direction generally perpendicular to a line extending from the midpoint of the front waist edge to the midpoint of the rear waist edge.

“Longitudinal”, with respect to a pant and its wearer, refers to the direction generally parallel with the wearer's standing height, or the vertical direction when the wearer is standing.

“Longitudinal” is also the direction generally parallel to a line extending from the midpoint of the front waist edge to the midpoint of the rear waist edge.

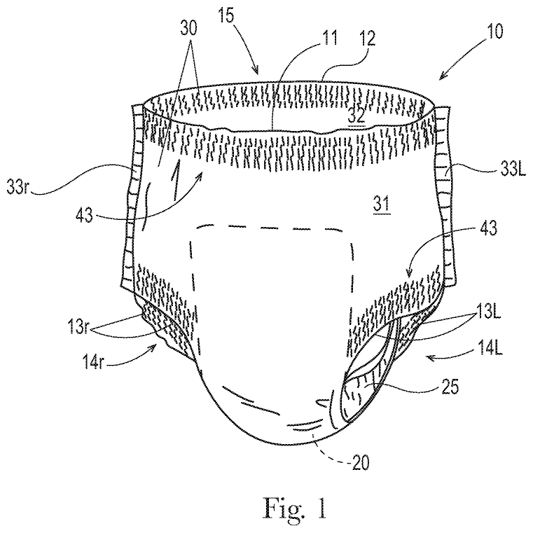

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and, preferably, to be recycled, composted or otherwise disposed of in an environmentally compatible manner). The pull-on garment is also preferably “absorbent” to absorb and contain the various exudates discharged from the body. A preferred embodiment of the absorbent article is the disposable absorbent pull-on garment, shown in FIG. 1.

The term “substrate” is used herein to describe a material that is primarily two-dimensional (i.e., in an XY plane) and whose thickness (in a Z direction) is relatively small (i.e. 1/10 or less) in comparison to its length (in an X direction) and width (in a Y direction). Non-limiting examples of substrates include a web, layer or layers of fibrous materials, nonwovens, and films and foils, such as polymeric films or metallic foils, for example. These materials may be used alone or may comprise two or more layers laminated together. As such, a web may be a substrate or may be a laminate of two or more substrates.

As used herein “homopolymer” means a polymer resulting from the polymerization of a single monomer, i.e., a polymer consisting essentially of a single type of repeating unit.

As used herein, the term “copolymer(s)” refers to polymer(s) formed by the polymerization of at least two different monomers. For example, the term “copolymer” includes the copolymerization reaction product of a monomer such as propene or butene, preferably 1-butene and an alpha-olefin, such as for example, ethylene, 1-hexene or 1-octene.

As used herein, the term “propene copolymer” or “propylene copolymer” means a copolymer of greater than 40 or 50 wt. % or more propene and at least one monomer selected from the group including ethylene and a C₄ to C₂₀ α-olefin.

As used herein, the term “butene copolymer” means a polymer of n-butene (1-butene) or 2-butene and at least one monomer selected from the group of C₂₋₃ and C₅₋₂₀ alpha olefins. Butene copolymers typically comprise a minimum amount at least about 40 or about 50 wt. % or more of a butene monomer such as 1-butene.

The term “heterophase” polymer means a polymer having an amorphous character and at least some substantial crystalline content (at least 5 wt. %, 10 wt. %, 20 wt. %, 40 wt. % or 50 wt. % crystalline content) that can provide cohesive strength in the cooled adhesive mass. The crystalline content can be in the form of stereoregular blocks or sequences.

The term “amorphous” means the substantial absence of crystallinity, (i.e.) less than 5% and less than 1%.

The term “sequence or block” means a polymer portion of repeating monomer that is similar in composition, crystallinity or other aspect.

As used herein, the term “open time” means the amount of time elapsed between application of a molten hot melt adhesive composition to a first substrate, and the time when useful tackiness or wetting out of the adhesive on a substrate effectively ceases due to solidification of the adhesive composition. Open time is also referred to as “working time.”

As used herein, the term “substrate” means any item having at least a partially or fully solidified fiber or planar surface with which contact with a hot melt adhesive composition is intended. In some cases the same area, circle, bead, line, filament or dot of hot melt adhesive composition is contacted with two or more substrates for the purpose of creating an adhesive bond there between. In some such cases the substrates are part of the same item: for example, folded film or folded non-woven, two sides of a cardboard sheet folded over, wherein the two sides are adhesively bonded together. In other such cases the substrates are part of different items: for example, a plastic film that is adhesively bonded to a non-woven or cardboard sheet. The substrates can be impermeable, permeable, porous or nonporous.

As used herein, the term “substantially” means generally the same or uniform but allowing for or having minor fluctuations from a defined property, definition, etc. For example, small measurable or immeasurable fluctuations in a measured property described herein, such as viscosity, melting point, etc. may result from human error or methodology precision. Other fluctuations are caused by inherent variations in the manufacturing process, thermal history of a formulation, and the like. The adhesive compositions of the, nonetheless, would be said to be substantially having the property as reported.

As used herein, the term “major proportion” means that a material or monomer is used at greater than 50 wt. %. As used herein, the term “primary component” means that a material or monomer is the more common substance or has the higher concentration in the mixture or polymer compared to others but may not be as much as 50 wt. %.

The transitional phrase “consisting essentially of” limits the scope of a claim to the specified materials but includes those that do not materially affect the basic and novel characteristics of the claimed materials. These characteristics include open time, cohesive strength (tensile strength), peel strength and viscosity. Meaningful amounts of a third polymer or amounts of a tackifier materially affect the basic and novel characteristics of the claimed materials.

### Article

Many existing absorbent pants are structured such that a backsheet and topsheet of a central chassis structure extend to, and from, the front and rear waist edges of the pant in the regions near the wearer's navel in the front, and small of the back in the rear. Separate and discrete side/hip panels are joined to longitudinal (side) edges of the central chassis structure in its front and rear regions, joining them to form the pant structure.

An alternate configuration for absorbent pants is one in which the central chassis structure does not extend to, or form, the front and rear waist edges of the pant. Rather, an elasticized belt structure entirely encircles the wearer's waist and forms the waist edge about the entire pant, and the side/hip panels. The central chassis is joined to the belt structure, usually on the inside thereof, with its ends disposed at locations in the front and rear waist regions somewhat below the waist edges of the belt structure. The elastic belt is usually relatively wide (in the longitudinal direction) and elastically stretchable in the lateral direction. It entirely encircles the wearer's waist, and thereby covers a relatively large amount of the wearer's skin. This configuration is sometimes known as a “belt” or “balloon” configuration (hereinafter, “belt” configuration).

FIG. 1 is a general simplified perspective depiction of a disposable absorbent pant 10 having a belt configuration. Pant 10 may include a central chassis 20 and a belt structure 30. Belt structure 30 may be elastically extensible in the lateral direction, providing elastic stretchability for ease of donning, and a snug and comfortable fit following donning. Central chassis 20 may include a wearer-facing, liquid permeable topsheet (not specifically shown in FIG. 1), an outer- or garment-facing backsheet (not specifically shown in FIG. 1) and an absorbent core (not specifically shown in FIG. 1) sandwiched or enveloped between the topsheet and backsheet. A pair of laterally opposing, longitudinally extending barrier cuffs 25 also may be included with the central chassis in a crotch region thereof, disposed adjacent to the topsheet. Generally the central chassis and barrier cuffs may have any construction and components, including leg cuff structures, suitable for disposable diapers, training pants, and adult incontinence pants, such as, but not limited to, those described in U.S. Pat. No. 8,939,957 and application(s) claiming priority thereto. Belt structure 30 may have a front portion 31 and a rear portion 32. Front and rear portions 31, 32 may be joined together at respective left and right side seams 33l, 33r. Belt structure 30 may form front and rear waist edges 11, 12 defining waist opening 15, and at least portions of left and right leg opening edges 13l, 13r of the pant 10. As shown in the FIG. 1, the portions of left and right leg opening edges 13l, 13r that the belt structure 30 forms may follow curvilinear paths. Alternatively, the portions of left and right leg opening edges 13l, 13r that the belt structure 30 forms may be substantially straight.

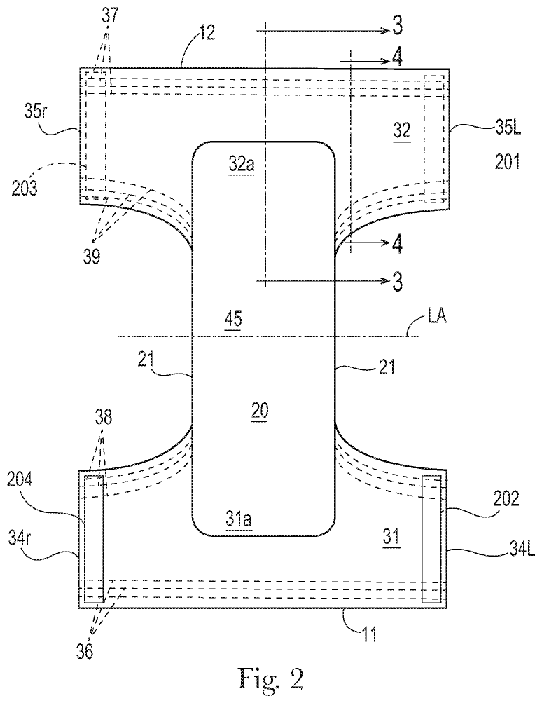

FIG. 2 is a simplified plan view of the precursor structure of the pant 10 shown in FIG. 1, shown prior to joining of front and rear portions 31, 32 along their respective side edges 34l, 35l and 34r, 35r. Front region 31a, including front portion 31, and rear region 32a, including rear portion 32, may each include anywhere from 25 percent to 40 percent of the overall longitudinal length of the precursor structure; correspondingly, a crotch region 45 may include anywhere from 20 percent to 50 percent of the overall longitudinal length of the precursor structure, with at least a portion thereof lying at lateral axis LA. The length of the side edges 34l and 34r may be about equal to the length of the side edges 35l and 35r respectively. The length of the side edges 34l and 34r may be substantially shorter than the length of the side edges 35l and 35r respectively. To form pant 10, the precursor structure may be folded along lateral axis LA to bring front and rear regions 31a, 32a, and front and rear portions 31, 32 together such that their side edges 34l, 35l and 34r, 35r, respectively, may be joined at seams 33l, 33r (as shown in FIG. 1). Seams 33l, 33R may be formed by adhesive, thermal, pressure, or ultrasonic bonding, and combinations thereof. These seams, and any other seams in the article, may be adhesively bonded by the substantially tackifier-free adhesives described herein. The length of the seams 33l, 33R may be about equal to the length of the side edges 34l, 35l, 34r and 35r. The length of the seams 33l, 33R may be substantially shorter than the length of the side edges 34l, 34r, 35l or 35r. In an alternative example, the seams may be formed by mechanical fasteners such as cooperating pairs of hook-and-loop fastening components disposed along side edges 34r, 35r and 34l, 35l. Fasteners may also include tape tabs, interlocking fasteners such as tabs & slots, buckles, buttons, snaps, and/or hermaphroditic fastening components. Exemplary surface fastening systems are disclosed in U.S. Pat. Nos. 3,848,594; 4,662,875; 4,846,815; 4,894,060; 4,946,527; 5,151,092; and 5,221,274, while an exemplary interlocking fastening system is disclosed in U.S. Pat. No. 6,432,098. The fastening system may also include primary and secondary fastening systems, as disclosed in U.S. Pat. No. 4,699,622. Additionally exemplary fasteners and fastener arrangements, the fastening components forming these fasteners, and the materials that are suitable for forming fasteners are described in U.S. Published Application Nos. 2003/0060794 and 2005/0222546 and U.S. Pat. No. 6,428,526.

Still referring to FIG. 2, one or both of front and rear portions 31, 32 may include at least a first elastic member 36, 37 disposed nearer the waist edges 11, 12 and at least a second elastic member 38, 39, disposed nearer the leg opening edges 13l, 13r. As suggested in FIG. 2, one or a plurality of waist elastic members 36, 37 may be disposed in a substantially straight lateral orientation, and one or a plurality of leg elastic members 38, 39 may be disposed along curvilinear paths to provide hoopwise elastic stretch about the leg openings 13l, 13r (as shown in FIG. 1). Alternatively, leg elastic members 38, 39 may be disposed in a substantially straight lateral orientation near the leg openings 13l, 13r. For purposes of manufacturing a pant having a neat appearance as will be described below, it may be desired that leg elastic members 38, 39 terminate proximate the respective longitudinal edges 21 of chassis 20. For purposes herein, where used to describe a positional relationship between two features, “proximate” is intended to mean within 2.0 cm, more preferably within 1.0 cm, of the identified features.

Elastic members 36, 37, 38 and 39 may be in the form of film or sections or strips thereof, strips, ribbons (flat strands), bands or strands of circular or any other cross-section, formed in any configuration of any elastomeric material such as described in, for example, co-pending U.S. application Ser. Nos. 11/478,386 and 13/331,695, and U.S. Pat. No. 6,626,879. A suitable example is LYCRA HYFIT strands, a product of Invista, Wichita, Kans. The elastic strands can have a cross section perpendicular to the strand longitudinal axis that is substantially non-circular. Substantially non-circular means that the ratio of the longest axis of the cross section to the shortest axis of the cross section is at least about 1.1. The ratio of the longest axis of the cross section to the shortest axis of the cross section can be about 1.1, about 3.0, about 5.0, about 10.0, or about 50.0. In some embodiments, this ratio can be at least about 1.1, or at least about 3.0. The shape of the cross section perpendicular to the strand longitudinal axis of the substantially non-circular strands can be rectangular (e.g., with rounded corners) which are also referred to as “flat” strands, trilobal, or oblong (e.g., oval) in the cross section. These substantially non-circular strands can provide more surface area to bond with nonwoven fabrics than the strands that are circular in cross section. Such an increase in surface area can increase the bond strength between the elastomeric strands and nonwoven. Flat elastic strands can be made of Spandex, Rubber, elastic Polyolefins, Styrenic block copolymers, Thermoplastic Polyurethane, Thermoplastic polyester, Polyether block amide or any combination of them. Thermoplastic polyurethane tapes available from Fulflex such as Clear-Fit′ can be used as flat elastic strands.

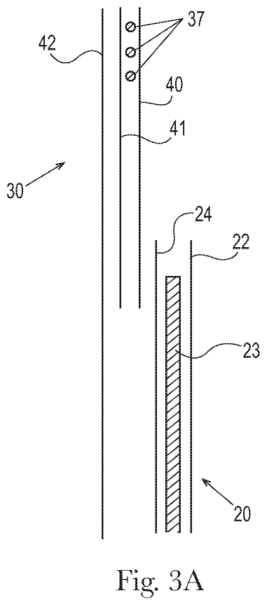

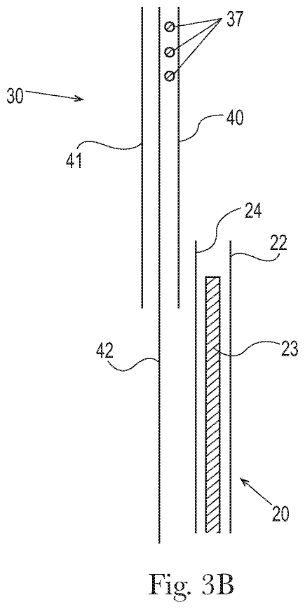

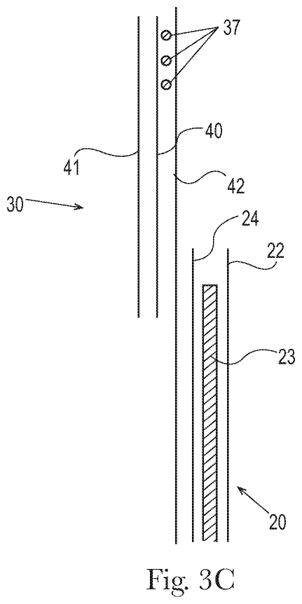

FIGS. 3A-3C are examples of potential longitudinal cross-sections taken at line 3-3 through the rear portion 32 of the belt structure and rear region of the pant as shown in FIG. 2, depicting features in three possible configurations. It can be appreciated that in each of these particular examples, the cross-section may substantially mirror a cross-section taken through the front portion 31 of the belt structure and the front region of the pant.

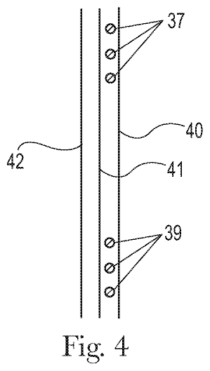

FIG. 4 is an example of a potential longitudinal cross-section taken at line 4-4 through the rear portion 32 of the belt structure and rear region of the pant as shown in FIG. 2, depicting features in one configuration. It can be appreciated that this cross-section may also be a substantial mirror image of a cross-section taken through the front portion 31 of the belt structure and the front region of the pant. Belt structure 30 where shown in FIG. 4 has the same layers and components as those depicted in FIG. 3A, but with the addition of leg elastic members 39 and without the chassis components, as a result of the location of the cross-section. As suggested in FIG. 2, leg elastic members 39 may terminate proximate the longitudinal edges 21 of central chassis 20; thus, they do not appear in FIGS. 3A and 3B. Additional elastics (not shown) may be disposed longitudinally between the waist elastics and the leg elastics.

Referring to FIGS. 3A-3C, chassis 20 may have liquid permeable topsheet 22 forming at least a portion of its inner, wearer-facing surface. Topsheet 22 may be formed of a nonwoven web material which is preferably soft and compatible with sensitive skin, and may be formed of and have any of the features of topsheets used in disposable diapers, training pants and inserts including those described in, for example, U.S. application Ser. No. 12/841,553. Chassis 20 may also have an outward-facing backsheet 24, which may be liquid impermeable. Backsheet 24 may be formed of and have any of the features of backsheets used in disposable diapers and training pants including those described in, for example, the U.S. patent application referenced immediately above. Chassis 20 may also have an absorbent core 23 disposed between topsheet 22 and backsheet 24. Absorbent core 23 may include one or more absorbent acquisition, distribution and storage material layers and/or components; it may be formed of and have any of the features of absorbent cores used in disposable diapers and training pants including those described in, for example, the U.S. patent application referenced immediately above.

As suggested in FIGS. 3A-3C, chassis 20 may be affixed to a belt structure 30, to the inner, wearer-facing side thereof, or alternatively, to the outer, garment-facing surface thereof. Chassis 20 may be bonded to the belt structure 30 by adhesive (such as by the substantially tackifier-free adhesives described herein), by thermal bonds/welds, mechanical fasteners or a combination thereof.

Referring to FIGS. 3A and 4, belt structure 30 may have a first belt layer 40 (or inner belt) which may be formed of a suitable nonwoven web material. Since the first belt layer may come into direct contact with the wearer's skin, it may be deemed preferable to select a nonwoven web material for the layer that is soft, comfortable and relatively breathable/vapor permeable. One or more waist elastic members 37 may be disposed between first belt layer 40 and a second belt layer 41 (also called the outer belt). Second belt layer 41 may be formed of the same, similar or differing nonwoven web material as first belt layer 40. First belt layer 40 and second belt layer 41 may be bonded together by adhesive (such as the substantially tackifier-free adhesives described herein), a pattern of thermal bonds or a combination thereof, such that first belt layer 40 and second belt layer 41 form a laminate, with the one or more waist elastic members 37 sandwiched and affixed there between. Similarly, referring to FIG. 4, the one or more leg elastic members 39 may be affixed and sandwiched between first belt layer 40 and second belt layer 41.

Also as shown in FIGS. 3A-3C and 4, the belt structure may include a longitudinally extending wrapping layer 42. Wrapping layer 42 may form a layer that wraps not only about the front and rear portions of the belt structure, but also extends from the front portion, around and beneath the chassis 20 through the crotch region, and into the rear portion. The wrapping layer 42 may be formed of a single material web disposed as a layer of the rear portion 32 of the belt structure 30, the central chassis 20, and the front portion 31 of the belt structure 30. Wrapping layer 42 may be disposed so as to form an outer layer or outer cover of the belt structure 30 in the front and rear portions as suggested in FIGS. 3A and 4, an intermediate layer in the front and rear portions as suggested in FIG. 3B, or an inner layer as suggested in FIG. 3C. Wrapping layer 42 may be formed of any suitable nonwoven web material having desired properties of softness and mechanical strength. Wrapping layer 42 is optional. That is, there are embodiments in which there is no wrapping layer and the rear portion 32 of the belt and the front portion 31 of the belt, when the article is laid out flat, may be discrete and without a common layer. The only part connecting the two belts in such embodiments would be the center chassis.

In some embodiments, the inner belt nonwoven 40 and the outer belt nonwoven 41 end at waist edge, such as is depicted in a cross-section view of an article in FIG. 7A, In other embodiments, such as FIG. 7B, the outer belt nonwoven 41 is extended up and folded over the inside of the central chassis 20. In this embodiment, the edge of the inner belt nonwoven at the waist edge may be aligned to or away from the folding that the outer belt nonwoven forms. In yet another embodiment as shown in FIG. 7C, the outer belt nonwoven may not be folded over at the waist, even though the inner belt nonwoven 40 is folded over the central chassis. In this embodiment, the edge of the outer belt nonwoven at the waist edge may be aligned to the folding that the inner belt nonwoven forms.

Examples of suitable nonwoven web materials useful for forming any of layers 40, 41 and 42 are described in U.S. application Ser. No. 13/090,761. Some examples described above, as well as other examples not expressly described, may also be advantageous because they may lend themselves to relatively efficient manufacture.

In general terms, the belt structure comprises an inner layer that is in contact with the wearer's skin when the article is worn. This inner layer may be formed of an inner nonwoven web comprising an inner surface. According to different embodiments described above, the inner surface that is in contact with the wearer's skin may be the first belt layer (for example, see FIG. 3A) or may be the wrapping layer (for example, see FIG. 3C). The belt structure also comprises an outer layer formed of an outer nonwoven web comprising an outer surface. This outer surface is the outermost surface of the article. In some embodiments described above, the outer surface may be the wrapping layer (for example, see FIG. 3A) or may be the second belt layer (for example, see FIG. 3C).

In some embodiments, the rear belt portion may be offset from the front belt portion, i.e., the rear belt portion may have a longer longitudinal length than the front belt portion longitudinal length to allow better coverage on the wearer, such as is described in U.S. filing Ser. No. 11/197,203.

The article, in some cases the chassis, may have a liquid permeable topsheet forming at least a portion of its inner, wearer-facing surface. The topsheet may be formed of a nonwoven web material which is preferably soft and compatible with sensitive skin, and may be formed of and have any of the features of topsheets used in disposable diapers, training pants and inserts including those described in, for example, U.S. application Ser. No. 12/841,553. The chassis 20 may also have an outward-facing backsheet, which may be liquid impermeable. The backsheet may be formed of and have any of the features of backsheets used in disposable diapers and training pants including those described in, for example, the U.S. patent application referenced immediately above. Chassis 20 may also have an absorbent core disposed between the topsheet and backsheet. The absorbent core may include one or more absorbent acquisition, distribution and storage material layers and/or components; it may be formed of and have any of the features of absorbent cores used in disposable diapers and training pants including those described in, for example, the U.S. patent application referenced immediately above. The belt structure may be formed of layers of nonwoven web which respectively form inner and outer layers of the belt and the layers of nonwoven web may sandwich one or more elastic members such as a plurality of strands of an elastomeric material. Suitable nonwoven web materials and suitable elastic materials that may be useful in the present invention include those described in U.S. Ser. No. 14/726,812. As suggested in FIG. 2, the chassis 20 may be affixed to a belt structure 30, to the inner, wearer-facing side thereof. Chassis 20 may be bonded to the belt structure 30 by adhesive (such as by the substantially tackifier-free adhesives described herein), by thermal bonds/welds, mechanical fasteners or a combination thereof. The belt structure may be referred to as flaps. Some of these article components are discussed in more detail below.

### Belt Nonwovens

The fibrous structures of the present invention may be made by any suitable process known in the art.

Nonwoven webs can be formed by direct extrusion processes during which the fibers and webs are formed at about the same point in time, or by preformed fibers which can be laid into webs at a distinctly subsequent point in time. Example direct extrusion processes include but are not limited to: spunbonding, spunlaid, meltblowing, solvent spinning, electrospinning, carded, film fibrillated, melt-film fibrillated, air-laid, dry-laid, wet-laid staple fibers, and combinations thereof typically forming layers.

As used herein, the term “spunbonded fibers” refers to small diameter fibers, which are formed by extruding molten thermoplastic material as filaments from a plurality of fine, usually circular capillaries of a spinneret. Spunbond fibers are quenched and generally not tacky when they are deposited onto a collecting surface. Spunbond fibers are generally continuous.

As used herein, the term “meltblown fibers” means fibers formed by extruding a molten thermoplastic material through a plurality of fine, usually circular, die capillaries as molten threads or filaments into converging high velocity gas (e.g. air) streams, which attenuate the filaments of molten thermoplastic material to reduce their diameter. Thereafter, the meltblown fibers are carried by the high velocity gas stream and are deposited on a collecting surface to form a web of randomly disbursed meltblown fibers.

Example “laying” processes include wetlaying and drylaying. Example drylaying processes include but are not limited to airlaying, carding, and combinations thereof typically forming layers. Combinations of the above processes yield nonwovens commonly called hybrids or composites. Example combinations include but are not limited to spunbond-meltblown-spunbond (SMS), spunbond-carded (SC), spunbond-airlaid (SA), meltblown-airlaid (MA), and combinations thereof, typically in layers. Combinations which include direct extrusion can be combined at about the same point in time as the direct extrusion process (e.g., spinform and coform for SA and MA), or at a subsequent point in time. In the above examples, one or more individual layers can be created by each process. For instance, SMS can mean a three layer, ‘sms’ web, a five layer ‘ssmms’ web, or any reasonable variation thereof wherein the lower case letters designate individual layers and the upper case letters designate the compilation of similar, adjacent layers. The fibers in a nonwoven web are typically joined to one or more adjacent fibers at some of the overlapping junctions. This includes joining fibers within each layer and joining fibers between layers when there is more than one layer. Fibers can be joined by mechanical entanglement, by chemical bond or by combinations thereof.

In some embodiments, nonwoven fabric can be unbonded nonwoven webs, electrospun nonwoven webs, flashspun nonwoven webs (e.g., TYVEK™ by DuPont), or combinations thereof. These fabrics can comprise fibers of polyolefins such as polypropylene or polyethylene, polyesters, polyamides, polyurethanes, elastomers, rayon, cellulose, copolymers thereof, or blends thereof or mixtures thereof. The nonwoven fabrics can also comprise fibers that are homogenous structures or comprise bicomponent structures such as sheath/core, side-by-side, islands-in-the-sea, and other bicomponent configurations. For a detailed description of some nonwovens, see “Nonwoven Fabric Primer and Reference Sampler” by E. A. Vaughn, Association of the Nonwoven Fabrics Indus-3d Edition (1992).

In some examples, suitable non-woven fiber materials may include, but are not limited to polymeric materials such as polyolefins, polyesters, polyamide, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392 to Land, the disclosure of which is incorporated by reference herein. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. No. 6,645,569 to Cramer et al.; U.S. Pat. No. 6,863,933 to Cramer et al.; and U.S. Pat. No. 7,112,621 to Rohrbaugh et al.; and in co-pending U.S. patent application Ser. Nos. 10/338,603 and 10/338,610 by Cramer et al.; and Ser. No. 13/005,237 by Lu et al., the disclosures of which are incorporated by reference herein.

The nonwoven fabrics can include fibers or can be made from fibers that have a cross section perpendicular to the fiber longitudinal axis that is substantially non-circular. Substantially non-circular means that the ratio of the longest axis of the cross section to the shortest axis of the cross section is at least about 1.1. The ratio of the longest axis of the cross section to the shortest axis of the cross section can be about 1.1, about 1.2, about 1.5, about 2.0, about 3.0, about 6.0, about 10.0, or about 15.0. In some embodiments, this ratio can be at least about 1.2, at least about 1.5, or at least about 2.0. These ratios can be, for example, no more than about 3.0, no more than about 6.0, no more than about 10.0, or no more than about 15.0. The shape of the cross section perpendicular to the fiber longitudinal axis of the substantially non-circular fibers can be rectangular (e.g., with rounded corners) which are also referred to as “flat” fibers, trilobal, or oblong (e.g., oval) in the cross section. These substantially non-circular fibers can provide more surface area to bond to the elastomeric fiber than nonwoven fabrics with fibers that are circular in cross section. Such an increase in surface area can increase the bond strength between the elastomeric film and fibers.

### Bicomponent Materials

An approach to improving consumer perceptions of component materials involves forming a nonwoven web of “bicomponent” polymer fibers, by spinning such fibers, laying them to form a batt and then consolidating them by calender-bonding with a pattern, selected to provide visual effects. Such bicomponent polymer fibers may be formed by spinnerets that have two adjacent sections, that express a first polymer from one and a second polymer from the other, to form a fiber having a cross section of the first polymer in one portion and the second polymer in the other (hence the term “bicomponent”). The respective polymers may be selected so as to have differing melting temperatures and/or expansion-contraction rates. These differing attributes of the two polymers, when combined in a side by side or asymmetric sheath-core geometry, cause the bicomponent fiber products to curl in the spinning process, as they are cooled and drawn from the spinnerets. The resulting curled fibers then may be laid down in a batt and calender-bonded in a pattern. It is thought that the curl in the fibers adds loft and fluff to the web, enhancing visual and tactile softness signals.

Nonwoven webs can be made of bicomponent or multi-component fibers. One of the components of the fibers, preferably the outer component, may be a soft polymer, such as polyethylene or elastic polyolefin, elastic polyurethane. For example, in a sheath/core bi-component fiber, the sheath can be made of polyethylene while core can be made of polypropylene. Often, the individual components comprise polyolefins such as polypropylene or polyethylene, or their copolymers, polyesters, thermoplastic polysaccharides or other biopolymers. In some embodiments, a nonwoven may be a PE/PET (polyethylene/polyethylene terephthalate) core/sheath bicomponent material, wherein the core is the PET and the outer sheath is PE. Additional nonwovens appropriate for the belt structures are disclosed in U.S. Ser. No. 62/210,635.

### Topsheet

In one embodiment, the absorbent article may comprise a topsheet. The topsheet may be compliant, soft feeling, and non-irritating to the wearer's skin and may be elastically stretchable in one or more directions. Further, the topsheet may be liquid pervious, permitting liquids (e.g., menses, urine, and/or runny feces) to penetrate through its thickness. Various topsheets may also comprise a hydrophilic material, for example, which is configured to draw bodily fluids into an absorbent core of the chassis when these fluids are expelled from the body. A suitable topsheet may be manufactured from a wide range of materials, such as woven and nonwoven materials, apertured or hydroformed thermoplastic films, apertured nonwovens, porous foams, reticulated foams, reticulated thermoplastic films, and/or thermoplastic scrims, for example. Suitable apertured films may comprise those described in U.S. Pat. Nos. 3,929,135, 4,324,246, 4,342,314, 4,463,045, 5,006,394, 5,628,097, 5,916,661, 6,545,197, and 6,107,539.

Apertured film or nonwoven topsheets typically may be pervious to bodily exudates, yet non-absorbent, and have a reduced tendency to allow fluids to pass back through and rewet the wearer's skin. Suitable woven and nonwoven materials may comprise natural fibers, such as, for example, wood or cotton fibers, synthetic fibers, such as, for example, polyester, polypropylene, or polyethylene fibers, or combinations thereof. If the topsheet comprises fibers, the fibers may be spunbond, carded, wet-laid, meltblown, hydroentangled, or otherwise processed, for example, as is generally known in the art.

The topsheet may comprise a skin care lotion. Examples of suitable lotions include, but are not limited to, those described in U.S. Pat. Nos. 5,607,760; 5,609,587; 5,635,191; 5,643,588; and 5,968,025, and as described in U.S. Application No. 61/391,353, and as described in U.S. Pub. No. 2014-0257216. Beyond these compositions, the absorbent article may comprise soluble cyclodextrin derivatives such as those described in U.S. Pub. No. 2014/0274870.

Additionally, the topsheet of the present disclosure may be a tufted laminate web as disclosed in U.S. Pat. No. 7,410,683, and/or may be an apertured web as disclosed in PCT/CN2014/083769 having an international filing date of Aug. 6, 2014.

In one embodiment, the topsheet may comprise graphics such that depth perception is created as described in U.S. Pat. No. 7,163,528. In other embodiments, the topsheet may be an integrated acquisition layer and topsheet as described in U.S. Ser. No. 14/680,426 or 14/634,928.

### Backsheet

In one embodiment, the absorbent article may comprise a backsheet. The backsheet may be impervious, or at least partially impervious, to fluids or body exudates (e.g., menses, urine, and/or runny feces) and may be manufactured from a thin plastic film, although other flexible liquid impervious materials may also be used. The backsheet may prevent the body exudates or fluids absorbed and contained in an absorbent core of the absorbent article from wetting articles which contact the absorbent article, such as bedsheets, pajamas, clothes, and/or undergarments. The backsheet may comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). A suitable backsheet may comprise a polyethylene film having a thickness of from about 0.012 mm (0.5 mils) to about 0.051 mm (2.0 mils). Examples of polyethylene films are manufactured by Clopay Corporation of Cincinnati, Ohio, under the designation BR-120 and BR-121, and by Tredegar Film Products of Terre Haute, Ind., under the designation XP-39385.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m2 to about 35 g/m2. The backsheet can be typically positioned adjacent the outer-facing surface of the absorbent core and can be joined thereto. For example, the backsheet may be secured to the absorbent core by a uniform continuous layer of adhesive, a patterned layer of adhesive, or an array of separate lines, spirals, or spots of adhesive. Illustrative, but non-limiting adhesives, include adhesives manufactured by H. B. Fuller Company of St. Paul, Minn., U.S.A., and marketed as HL-1358J, or any of the substantially tackifier-free adhesives described herein. An example of a suitable attachment device including an open pattern network of filaments of is disclosed in U.S. Pat. No. 4,573,986. Another suitable attachment device including several lines of adhesive filaments swirled into a spiral pattern is illustrated by the apparatus and methods shown in U.S. Pat. Nos. 3,911,173; 4,785,996; and 4,842,666. Alternatively, the attachment device may include heat bonds, pressure bonds, ultrasonic bonds, dynamic mechanical bonds, or any other suitable attachment device or combinations of these attachment devices.

In one embodiment, the backsheet may be embossed and/or matte-finished to provide a more cloth-like appearance. Further, the backsheet may permit vapors to escape from the absorbent core of the absorbent article (i.e., the backsheet is breathable) while still preventing, or at least inhibiting, fluids or body exudates from passing through the backsheet. In one embodiment, the size of the backsheet may be dictated by the size of the absorbent article and the design or configuration of the absorbent article to be formed, for example.

### Absorbent Core

In various embodiments, the absorbent article may comprise an absorbent core (also referred to as an “absorbent member” or “absorbent assembly” or “absorbent structure” or “absorbent composite”) that is disposed between the topsheet and the backsheet. The absorbent core may comprise a laterally extending front edge in the front waist region, a longitudinally opposing and laterally extending back edge in the back waist region, a first longitudinally extending side edge, and a laterally opposing and second longitudinally extending side edge. Both of the side edges may extend longitudinally between the front edge and the back edge. In one embodiment, more than one absorbent core or more than one absorbent core layer may be provided in an absorbent article, for example. The absorbent core may be any suitable size or shape that is compatible with the absorbent article. Example absorbent structures for use as the absorbent core of the present disclosure that have achieved acceptance and commercial success are described in U.S. Pat. Nos. 4,610,678; 4,673,402; 4,888,231; and 4,834,735.

In one embodiment, suitable absorbent cores may comprise cellulosic airfelt material (also referred to as pulp). For instance, such absorbent cores may comprise less than about 40%, 30%, 20%, 10%, 5%, or even 1% of the cellulosic airfelt material as determined by weight. Additionally, such an absorbent core may be primarily comprised of an absorbent gelling material (AGM) in amounts of at least about 60%, 70%, 80%, 85%, 90%, 95%, or even about 100% as determined by weight. Furthermore, a portion of the absorbent core may comprise a microfiber glue (if applicable). Such absorbent cores, microfiber glues, and absorbent gelling materials are described in U.S. Pat. Nos. 5,599,335; 5,562,646; 5,669,894; 6,790,798; and 7,521,587 and in U.S. Pat. Publ. No. 2004/0158212.

In one embodiment, the core, including multiple layers making up the core system, may be printed and embossed as described in U.S. Pat. No. 8,536,401.

In one embodiment, the core may be separable from the chassis as disclosed in U.S. Pat. Nos. 6,989,006; 7,381,202; 7,175,613; 7,824,386; 7,766,887; and 6,989,005. In such embodiments, the measurements described in this disclosure may be made to the chassis alone or may be made to the chassis in combination with the separable core/absorbent assembly.

In one embodiment, the absorbent article of the present disclosure, and particularly, a portion where the absorbent member is disposed, may have a body fluid absorption rate greater than 3 g/sec according to U.S. Pat. No. 6,649,810. According to U.S. Pat. No. 6,649,810, the expression “the portion (of the absorbent article) where the absorbent member is disposed” is intended to mean the portion occupied by the absorbent member when the absorbent article is flatly unfolded and seen in its plan view.

In one embodiment, the absorbent structure may have an intake factor greater than 3 according to U.S. Pat. No. 7,073,373, wherein the intake factor is defined as the absorbent core permeability divided by the normalized retention capacity (which is defined by the Retention Capacity Test—also according to U.S. Pat. No. 7,073,373).

In one embodiment, the absorbent composite has a body fluid absorption greater than 75 g/100 cm2, according to U.S. Pat. No. 6,649,810.

In one embodiment, a target location of the absorbent article may have a wicking value greater than 36%, according to U.S. Pat. No. 6,383,960.

In one embodiment, the absorbent article may have a bending stiffness between 0.05-1.0 gf, according to U.S. Pat. No. 5,810,796.

In one embodiment, the absorbent article may have a crotch fluid absorption rate greater than 3 g/sec according to U.S. Pat. No. 6,649,810. In one embodiment, a freeze-dried composite of the absorbent composite may have an intake rate of at least about 1.9 cubic centimeters (cc) of liquid/second at 80% composite saturation according to U.S. Pat. No. 6,689,934.

In some embodiments the absorbent core may comprise channels as described in U.S. Pat. No. 8,568,566; U.S. Pub. Nos. 2012/316046, 2014/027066, 2014/163500, 2014/163506, 2014/163511, 2012/316526, 2012/316527, 2012/316528, 2012/316529, 2012/316523, 2014/163501, 2014/163502, 2014/163503 and European Pub. Nos. 2532328, 2532329, 2717823, 2717820, 2717821, 2717822, 2532332, 2740449, and 2740452.

In some embodiments the absorbent layer may comprise at least two channels substantially free of absorbent polymer particles extending through the thickness of the absorbent layer in the longitudinal dimension of the absorbent layer. By extending in the longitudinal dimension of the absorbent layer, it is meant that the channels extend essentially in the longitudinal dimension, i.e. they extend more in the longitudinal dimension than in the transverse dimension, e.g. at least twice as much in the longitudinal dimension than in the transverse dimension.

“Channels” as used herein refer to discrete portions of one or more of the absorbent layers of the absorbent core extending through the thickness of the absorbent layer which are substantially free of absorbent polymer (particles or fibers), i.e., no absorbent polymer particles are intentionally present in such a channel (longitudinal main channel or secondary channel) of an absorbent structure. However, it should be understood that, accidentally, a small, negligible amount of absorbent polymer particles may be present in the channel, which may not contribute to any significant degree to the overall functionality (e.g. absorbency of the absorbent structure). Typically, the channels possess two transverse edges (in the shortest dimension) and two longitudinal edges (in the longest dimension) running between the transverse edges. The transverse edges of the channels may be straight (i.e., perpendicular to the longitudinal side edges), angled or curved. The channels may have an average width w of at least 3 mm (the average of a channel is defined as the average distance between the longitudinal side edges) or may have at least 4% of the width of the absorbent layer.

The channels may be permanent. By permanent, it is meant that the integrity of the channels is at least partially maintained both in dry state and wet state, i.e., the channels are resistant to external forces caused by movements of the diaper's wearer. Permanent channels are obtained by immobilizing the absorbent polymer on the substrate layer, such as by applying a thermoplastic adhesive material over the absorbent layer. Any of the adhesives used in the core may be the substantially tackifier-free adhesives described herein. The absorbent layer of the present disclosure may comprise in particular permanent channels formed by bonding of a first substrate layer and a second substrate layer through the channels. Typically, glue may be used to bond both substrate layers through the channel, but it is possible to bond via other known means, for example ultrasonic bonding, pressure bonding or thermal bonding. The supporting layers can be continuously bonded or intermittently bonded within the channels.

In some embodiments, it may be desirable to have an array of articles comprising absorbent cores with channels, such as those disclosed in 62/104,330.

### Leg Cuffs

In one embodiment, the chassis of the absorbent article may comprise longitudinally extending and laterally opposing leg cuffs and that are disposed on the interior surface of the chassis that faces inwardly toward the wearer and contacts the wearer. The leg cuffs and may comprise one or more elastic gathering members disposed at or adjacent the proximal edge of one or both of the leg cuffs. In addition, the elastic gathering members of the leg cuff may also comprise one or more elastic strands disposed at or adjacent the distal edge of one or both of the leg cuffs. The elasticized leg cuffs may comprise several embodiments for reducing the leakage of body exudates or fluids in the leg regions. The elasticized leg cuffs are sometimes referred to as leg bands, barrier cuffs, elastic cuffs, or gasketing cuffs. Suitable elasticized leg cuffs may comprise those described in U.S. Pat. Nos. 3,860,003, 4,909,803, 4,695,278, 4,795,454, 4,704,115, and 4,909,803, and U.S. Pat. Publ. No. 2009/0312730. The leg cuffs may be formed by folding portions of the chassis laterally inward, i.e., toward the longitudinal axis, to form both the respective leg cuffs and the side edges of the chassis. In other embodiments, the leg cuffs may be formed by attaching an additional layer or layers to the chassis at or adjacent to each of the respective side edges of the chassis. In one embodiment, the chassis may also comprise other elastics disposed adjacent the side edges which may cause the article to form into a “U” shape when allowed to relax thereby pulling the interior surface of the front waist region toward the interior surface of the back waist region.

In one embodiment, each leg cuff may comprise a proximal edge. These edges are positioned proximate to the longitudinal axis compared to distal edges. The leg cuffs may overlap the absorbent core, i.e., the proximal edges lie laterally inward of the respective side edges and of the absorbent core. Such an overlapped configuration may be desirable in order to impart a more finished appearance to the absorbent article than that imparted by a non-overlapped configuration. In other embodiments, the leg cuffs may not overlap the absorbent core.

In one embodiment, each leg cuff may be attached to the interior surface of the chassis in a leg cuff attachment zone (not shown) adjacent to the front waist end edge and in a longitudinally opposing leg cuff attachment zone (not shown) adjacent to the back waist end edge. In one embodiment, between the leg cuff attachment zones, the proximal edge of the leg cuff remains free, i.e., not attached to the interior surface of the chassis or to the absorbent core. Also, between the longitudinally opposing leg cuff attachment zones, each leg cuff may comprise one or more (specifically including one, two, three, or four elastic strands per leg cuff) longitudinally extensible cuff elastic gathering members that may be disposed at or adjacent to the proximal edge of the leg cuff by any suitable methods. Each of such cuff elastic gathering members may be attached over the leg cuff's entire length or over only a portion of the leg cuff's length. For example, such cuff elastic gathering members may be attached only at or near the leg cuff's longitudinally opposing ends and may be unattached at the middle of the leg cuff's length. Such cuff elastic gathering members may be disposed in the crotch region and may extend into one or both of the front waist region and the back waist region. For example, an elastic gathering member may be attached at or adjacent to the proximal edge of each of the leg cuffs and extends into both the front waist region and the back waist region.

In various embodiments, each cuff elastic gathering member may be enclosed inside a folded hem for example. In various embodiments, the cuff elastic gathering members may be sandwiched between two layers forming the leg cuff, by two layers of the chassis, or may be attached on a surface of the chassis or the leg cuff and remain exposed.

In one embodiment, when stretched, the cuff elastic gathering member disposed adjacent to each leg cuff's proximal edge allows the leg cuff proximal edge to extend to the flat uncontracted length of the chassis, e.g., the length of the chassis. When allowed to relax, the cuff elastic gathering member contracts to pull the front waist region and the back waist region toward each other and, thereby, bend the article into a “U” shape in which the interior of the “U” shape may be formed by the portions of the article that are intended to be placed toward the body of the wearer (i.e., interior surface). Because each of the proximal edges remains free between the longitudinally oriented leg cuff attachment zones, the contractive force of the elastic gathering member may lift the proximal edge of the leg cuff away from the interior surface of the chassis. This lifting of the proximal edges when the article is in the relaxed condition lifts the leg cuffs into a position to serve as side barriers to prevent, or at least inhibit, leakage of bodily exudates.

Examples of acceptable leg cuffs are disclosed in U.S. Ser. No. 13/457,521, filed Apr. 27, 2012, including the configurations disclosed by FIGS. 8a-t of the '521 application.

### Waistband

In one embodiment, the article may comprise an elasticized waistband. The elasticized waistband may provide improved fit and containment and may be configured to elastically expand and contract laterally to dynamically fit a wearer's waist. The elasticized waistband may extend longitudinally from the waist edge of the absorbent article toward the waist edge of the absorbent core. In one embodiment, the absorbent article may have two elasticized waistbbands, one positioned in the back waist region and one positioned in the front waist region, although other pant embodiments may be constructed with a single elasticized waistband. The elasticized waistband may be constructed in a number of different configurations including those described in U.S. Pat. Nos. 4,515,595 and 5,151,092, and including the consolidated gathers as disclosed in U.S. Ser. Nos. 13/490,543, 13/490,548, and 13/490,554.

In one embodiment, the elasticized waistbands may comprise materials that have been “prestrained” or “mechanically prestrained” (i.e., subjected to some degree of localized pattern mechanical stretching to permanently elongate the material). The materials may be prestrained using suitable deep embossing techniques. In other embodiments, the materials may be prestrained by directing the material through an incremental mechanical stretching system as described in U.S. Pat. No. 5,330,458. The materials may then be allowed to return to their substantially untensioned condition, thus forming a zero strain stretch material that is extensible, at least up to the point of initial stretching. Examples of zero strain materials are disclosed in U.S. Pat. Nos. 2,075,189, 3,025,199, 4,107,364, 4,209,563, 4,834,741, and 5,151,092.

### Flaps

The flaps may be discrete from or integral with the chassis. A discrete flap is formed as separate element, which is joined to the chassis. In some embodiments this may include a front and/or back belt-like flaps (“belts”) being joined across the front and back (or rear) waist regions of the chassis, at least across end edges of the chassis. In some embodiments the waistbands can overlap the flaps to create a continuous belt-like structure.

The belt-like flaps may comprise an inner nonwoven layer and an outer nonwoven layer and elastics there between. The inner and outer nonwoven layers may be joined using adhesive or thermoplastic bonds. Various suitable belt-like flap configurations can be found in U.S. Pub. No. 2013-0211363.

An integral flap is a portion, one or more layers, of the chassis that projects laterally outward from the longitudinal edge. The integral flap may be formed by cutting the chassis to include the shape of the flap projection.

While many of the embodiments illustrated in this application having belt-like flaps are pant articles, taped articles may have belt-like flaps disposed in one or both waist regions as well.

The structure of flaps play an important role in the functionality of the absorbent article and are fundamentally different than the elastics used in underwear. In order to sustain the fit of the article even after loading the article comprises elastomeric element(s), including films and/or strands that are disposed proximate to and along the side seams of the article and extend laterally from one side toward the other. These elastomeric element(s) should create a normal force against the body sufficient to anchor the article. The location of the elastomeric element(s), as well as the forces exerted by the elastomeric element(s) can be varied to ensure proper anchoring at the hips and along the body specifically across the front waist region and in the back waist region. One form of anchoring beneficial for sustaining the fit of a loaded article is disclosed in U.S. Pat. No. 5,358,500 Absorbent Articles Providing Sustained Dynamic Fit issued Oct. 25, 1994 to LaVon, et al.

The seams may each be from about 70 mm to about 200 mm, from about 100 mm to about 190 mm, or from about 130 mm to about 150 mm. The seams are the portions of the flap that overlap (i.e., the distance from the waist opening to the leg opening of the overlapped or abutted flaps).

### Fastening System

The absorbent article may also include a fastening system. When fastened, the fastening system interconnects the front waist region and the rear waist region resulting in a waist circumference that may encircle the wearer during wear of the absorbent article. The fastened elements connecting the front and back waist regions form refastenable side seams. This may be accomplished by flaps in the back waist region interconnecting with flaps in the front waist region or by flaps in the back waist region interconnecting with the chassis in the front waist region. The fastening system may comprises a fastener such as tape tabs, hook and loop fastening components, interlocking fasteners such as tabs & slots, buckles, buttons, snaps, and/or hermaphroditic fastening components, although any other known fastening means are generally acceptable. The fasteners may releasably engage with a landing zone, which may be a woven or nonwoven. Some exemplary surface fastening systems are disclosed in U.S. Pat. Nos. 3,848,594; 4,662,875; 4,846,815; 4,894,060; 4,946,527; 5,151,092; 5,221,274. Particularly, the flaps may be configured as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of the '065 application). An exemplary interlocking fastening system is disclosed in U.S. Pat. No. 6,432,098. The fastening system may also provide a means for holding the article in a disposal configuration as disclosed in U.S. Pat. No. 4,963,140. The fastening system may also include primary and secondary fastening systems, as disclosed in U.S. Pat. No. 4,699,622. The fastening system may be constructed to reduce shifting of overlapped portions or to improve fit as disclosed in U.S. Pat. Nos. 5,242,436; 5,499,978; 5,507,736; and 5,591,152.

In some embodiments, a refastenable system may be used such as those disclosed in U.S. application Ser. Nos. 13/929,900, 13/929,970. The particular hooks (types and sizes) and landing zones disclosed in 62/063,445 may also be used.

The embodiment shown in FIG. 2 comprises fastening elements 201-204 that may be refastenably joined together. Particularly, fastening elements 201 and 203 may be hook elements that join with fastening elements 202 and 204, respectively. Fastening elements 201 and 203 are shown on an exterior surface of the elasticized belt 30, but they may also be placed on an interior surface of the elasticized belt 30. Fastening elements 202 and 204 may be a discrete member of loop elements or may be an area of loop elements that is part of a nonwoven sheet lining the interior (as shown) or exterior of the elasticized belt. In another embodiment, fastening elements 201 and 203 may be loop elements and fastening elements 202 and 204 may be hook elements.

It is understood that when the fastening elements 201-204 mate interior surface to interior surface of the elasticized belt 30, a flange seam is formed. This may be a permanent side seam. One embodiment is shown in FIG. 5. FIG. 5 shows the rear portion inner belt nonwoven 40 coming together with the front portion inner belt nonwoven 400, looking at the article from the top. The interiors of both inner belts are bonded, forming a permanent seam. Both the front and rear portion inner belts 40 and 400 may be made of PP (polypropylene), so the PP to PP seam provides a bond with good strength. Both front and rear outer belts 41 and 410 may be made of PE/PET (polyethylene/polyethylene terephthalate) core/sheath bicomponent material, wherein the core is the PET and the outer sheath is PE.

When the fastening elements 201-204 mate interior surface to exterior surface of the elasticized belt 30, an overlap seam is formed. An example of this is shown in FIG. 6. FIG. 6 shows a top view of the area where the rear belt 32 is overlapped by the front belt 31, forming a refastenable seam. Specifically, the rear portion outer belt nonwoven 41 is overlapped by, ie., brought together with, the front portion inner belt nonwoven 400. The rear portion outer belt nonwoven may be a blend of PE/PET (as described above), while the front portion inner belt nonwoven may be made of PP. In FIG. 6, the rear portion outer belt nonwoven has an area comprising hooks 500. In general, the hooks may be disposed on the outer surface of a belt and face away from the wearer and connect into the inner layer of the opposing belt, or connect into a landing zone disposed on the inner layer of the opposing belt. In some cases, the hooks may be attached to the nonwoven with more than one type of bonding, for example, an adhesive (such as the substantially tackifier-free adhesives described herein) plus pressure bonding. In some cases, the bonding may be effective to bond the hooks 500, the outer belt nonwoven 41 and the inner belt nonwoven 40 to assure that the force exerted on the hook 500 does not overcome the bond between the hook and the outer belt nonwoven, and the bond between the outer belt nonwoven and the inner belt nonwoven. In FIG. 6, the polypropylene of the front portion inner belt nonwoven may provide a good landing zone, where the hooks directly connect with the fibers of the front portion inner belt nonwoven. Or, there can be an additional landing zone with loops, where the landing zone is attached to the front portion inner belt nonwoven.

The front or rear belt portion that is used as the landing zone may also have an area of additional bonding between its outer and inner belts to assure adequate strength as a landing zone. That is, a front or rear belt portion that is acting as a landing zone must have a strong enough bond between its inner and outer belts to hold when the front or rear belt portion is attached to the opposite belt portion. Therefore, as shown in FIG. 6, there is bonding 502 between the inner and outer belts of the front belt portion. There may be additional bonding (e.g., more adhesive, higher ultrasonic frequency bonding, and/or more pressure bonding, etc.) for a length from about 10 mm to about 45 mm, D1, to assure that the force exerted on the front belt portion as a landing zone does not overcome the bond between the front belt portion's inner and outer belts. The bonding between the front belt portion inner and outer belts may be less outside of the length of D1, as, in general, less bonding allows the material to be softer.

The fastening elements 201-204, first and second fastening elements 202 and 204 and first and second mating fastening elements 201 and 203, may be fastened during the manufacturing process and/or fastened in the package prior to use by the wearer or caregiver (i.e., the pant may be sold in “closed form”). Alternatively, the pant may be sold in “open form,” where the fastening elements 201-204 are present but are not joined in the package.

### Adhesive

The belted structures of the present invention may comprise hot melt adhesive material, used to bond various substrates. The hot melt adhesives may be made with substantially less than 40 wt. %, less than 20 wt. % or be substantially free of an effective amount of a conventional tackifier material that can add any aspect of open time, substrate wetting or tack to the adhesive material, ie., be substantially tackifier-free. Common hot melt adhesives are made by combining polymer and additive components in a substantially uniform thermoplastic blend.

In some embodiments, the adhesive composition may comprise a first amorphous polymer and a second heterophase polymer. The amorphous polymer comprises an amorphous or random polymer comprising an alpha olefin co-polymer comprising major proportion of propene. The second polymer comprises a heterophase alpha olefin-co-polymer having amorphous character and at least some substantial crystalline content. The crystalline content can be in the form of one or more polymer blocks or sequences that are stereoregular. In one embodiment, these sequences or blocks are substantially crystallizable sequences or blocks. The adhesive material may comprise a first polymer comprising a polyolefin comprising a substantially amorphous or randomly polymerized polymer material and a second polymer comprising a heterophase polymer.

In some embodiments, the adhesive material may comprise a first polymer comprising a polyolefin copolymer comprising a substantially amorphous or randomly polymerized polymer material comprising 1-butene and a second amorphous polymer comprising a compatible amorphous liquid butene polymer such as a polyisobutylene polymer or similar material. The polyisobutylene polymer may comprise a substantial proportion (greater than 50 mole % and often greater than 90 mole %) of an isobutylene monomer.

The first amorphous polymer may comprise typically butene (e.g.) 1-butene, and can be a copolymer or terpolymer that can contain ethylene, propene or a second C₄₋₄₀ olefin polymer. These substantially amorphous low crystallinity polymers have less than 10% and preferably less than 5% crystalline character.

The second heterophase olefin polymer comprises a first poly alpha olefin polymer comprising a substantial proportion (greater than 40 or 50 mole %) of a propene monomer and comprises an amorphous polymer with some crystalline content.

The amorphous polymer is a butene-based copolymer (the minimum amount is at least about 30 or 40 or 50 or 60 wt. % of 1-butene), which may also be referred to as a random butene-α-olefin copolymer. The butene copolymer includes one or more units, i.e., monomer units, derived from propene, one or more comonomer units derived from ethylene or α-olefins including from 4 to about 20 carbon atoms.

The first copolymer comprises about 30 mole %-about 75 mole %, preferably about 40 mole % to about 70 mole %, about 50 mole %-about 65 mole %, of units derived from butene. In addition to butene-derived units, the present copolymer contains from about 70 mole %-about 30 mole % to about 60 mole %-about 40 mole %, of units derived from preferably ethylene, propene or at least one C_{5 to 10} alpha-olefin monomer.

In one or more embodiments, the alpha-olefin comonomer units can also be derived from other monomers such as ethylene, 1-butene, 1-hexane, 4-methyl-1-pentene and/or 1-octene. Exemplary alpha-olefins are selected from the group consisting of ethylene, butene-1, pentene-1,2-methylpentene-1,3methylbutene-1, hexene-1,3-methylpentene-1,4-methylpentene-1,3,3-dimethylbutene-1, heptene-1, hexene-1, methylhexene-1, dimethylpentene-1, trimethylbutene-1, ethylpentene-1, octene-1, methylpentene-1, dimethylhexene-1, trimethylpentene-1, ethylhexene-1, methylethylpentene-1, diethylbutene-1, propylpentane-1, decene-1, methylnonene-1, nonene-1, dimethyloctene-1, trim ethylheptene-1, ethyloctene-1, methylethylbutene-1, diethylhexene-1, dodecene-1, and hexadodecene-1.

In one or more embodiments, amorphous copolymer comprises about 30 mole %-about 75 mole %, preferably about 40 mole % to about 60 mole % of units derived from butene and from about 70 mole %-about 30 mole % to about 60 mole %-about 40 mole %, about 50 mole %-about 65 mole %, of units derived from at least one alpha-olefin monomer selected from ethylene, propene, 1-hexene or 1-octene. Small amounts of α-olefin monomer(s) can be used in the range of about 0.1 to 20 mole %. The amorphous polymer has a weight average molecular weight (Mw) of about 1,000 to about 25,000 or less, or about 2,000 to 20,000, or from about 5000 to about 45,000.

In one or more embodiments, first copolymer comprises about 30 mole %-about 70 mole %, or about 40 mole % to about 60 mole % of units derived from butene and from about 70 mole %-about 30 mole % to about 60 mole %-about 40 mole %, of units derived from propene, while small amounts of α-olefin monomer(s) can be used in the range of about 0.1 to 20 mole %.

The amorphous polymer may have a weight average molecular weight (Mw) of about 1,000 to about 50,000 or less, or about 5,000 to 45,000.

The amorphous copolymer may have a viscosity of less than 10,000 mPa·s (1 centipoise [cps]=1 mPa·s), for example about 2000 to 8000 mPa·s, when measured by ASTM D3236 at 190° C. Melt Viscosity was determined according to ASTM D-3236, which is also referred to herein as “viscosity” and/or “Brookfield viscosity”.

Some examples of amorphous polyolefin include the Rextac polymers made by Huntsman including Rextac E62, E-63, E-65, 2815, 2830, etc. See, for example Sustic, U.S. Pat. No. 5,723,546 for a description of the polymers and which is expressly incorporated herein. Other useful amorphous polymers are sold as Vestoplast® and Eastoflex® materials.

The adhesive material may comprise a second polyolefin comprising a substantially heterophase copolymer. The heterophase polyolefin may comprise a propene copolymer (i.e.) propene-based polymer with other comonomer(s). The propene-based polymer backbone preferably comprises propene and one or more C₂ or C₄₋₂₀ α-olefins. The propene-based heterophase polymer, for example, may comprise propene and ethylene, hexene or optionally other C₂ or C₄₋₂₀ α-olefins. The polymer comprises about 99.5 to about 70 wt. %, preferably about 95 to about 75 wt. % of units derived from propene. In addition to propene derived units, the present copolymer contains from about 0.1 to 30 wt. % preferably from about 5 to 25 wt. %, of units derived from preferably at least C₂₋₄ or a C₅₋₁₀ alpha-olefin.

In one or more embodiments, the second copolymer comprises a major proportion of propene and about 0.1 to 30 wt. %, or 2 to 25 wt. % ethylene. In one or more embodiments, the second copolymer comprises a major proportion of propene and about 0.1 to 30 wt. %, or 2 to 25 wt. % 1-butene.

In one or more embodiments, the second copolymer comprises a major proportion of propene and about 0.1 to 30 wt. %, or 2 to 25 wt. % 1-hexene. In one or more embodiments, the second copolymer comprises a major proportion of propene and about 0.1 to 30 wt. %, or 2 to 25 wt. % 1-octene.

Other comonomer for use in either the first or second polyolefin comprise ethylene or α-olefins containing 4 to 12 carbon atoms. Exemplary α-olefins may be selected from the group consisting of ethylene; 1-butene; 1-pentene; 2-methyl-1-pentene; 3-methyl-1-butene; 1-hexene-3-methyl-1-pentene-4-methyl-1-pentene-3,3-dimethyl-1-butene; 1-heptene; 1-hexene; 1-methyl-1-hexene; dimethyl-1-pentene; trimethyl-1-butene; ethyl-1-pentene; 1-octene; methyl-1-pentene; dimethyl-1-hexene; trimethyl-1-pentene; ethyl-1-hexene; 1-methylethyl-1-pentene; 1-diethyl-1-butene; propyl-1-pentene; 1-decene; methyl-1-nonene; 1-nonene; dimethyl-1-octene; trimethyl-1-heptene; ethyl-1-octene; methylethyl-1-butene; diethyl-1-hexene; 1-dodecene and 1-hexadodecene. Preferred C₄₋₁₀ alpha-olefins are those having 6 to 8 carbon atoms, with the most preferred alpha-olefin being 1-hexene and 1-octene.

Preferred propene copolymers are copolymers wherein the comonomer is ethylene, 1-butene, 1-hexene or 1-octene. The stereo-regular (isotactic or syndiotactic) sequence or block content of the polymers imparts a heterophase (partial amorphous and partial crystalline) character of crystallizable content to the polymers. As used herein and as applied to semi-crystalline heterophase copolymers, the term “crystallizable” describes those polymer sequences or blocks that can crystallize upon cooling. Crystalline content of the solidified semicrystalline copolymers increases the cohesive strength of the hot melt adhesives. Hot melt adhesive formulations based on metallocene polymerized semicrystalline copolymers can eventually build sufficient crystalline content over time to achieve good cohesive strength in the formulation.

The second heterophase polymer comprises crystallizable polymer blocks or sequences, preferably of stereoregular sequences of polymerized monomer such as ethylene or propene, which sequences are long enough to crystallize, typically at least repeating or block monomer units per sequence.

In preferred embodiments, the crystallizable segments can be stereoregular or isotactic. Isotacticity of the olefin sequences can be achieved by polymerization with the choice of a desirable catalyst composition. The Isotacticity is conventionally measured using DSC or C-13 NMR instrumental techniques.

The heterophase polymer has a crystallinity of at least 5 wt. %, 10 wt. %, 20 wt. %, 40 wt. % or 50 wt. %, preferably between 20% and 80%, more preferably between 25% and 70%.

The heat of fusion of the heterophase copolymers (by ASTM E793) is about 10 J/g to about 70 J/g and about 15 J/g to about 70 J/g, with a melting point less than 150° C. and about 105° C. to about 135° C.

The heterophase polymer has a weight average molecular weight (Mw) of about 20,000 or less, preferably about 10,000 or less, preferably about 500 to 8,000.

The heterophase copolymer has a viscosity of less than 20,000 mPa·s (1 centipoise [cps]=1 mPa·s), for example less than 15000 mPa·s, in certain application less than 10,000 mPa·s and less than 5,000 mPa·s when measured at 190° C. using a Brookfield viscometer (as measured by ASTM D 3236) which is also referred to herein as “viscosity” and/or “Brookfield viscosity.”

Some examples of heterophase polymers useful in the hot melt adhesive compositions of include polyolefin such as polyethylene, polypropylene, and copolymers thereof such as polypropylene based elastomers sold by ExxonMobil Chemical of Houston, Tex. under the trade name VISTAMAXX™ and polyethylene based elastomers such as those sold by Dow Chemical Company of Midland, Mich. under the trade names AFFINITY™ and ENGAGE™.

Other heterophase polymers that are useful in the hot melt adhesive compositions include the polyolefin elastomers VISTAMAXX™ 8816, VISTAMAXX™ 2230, and ENGAGE™ 8200. AFFINITY™ GA 1900 has a density of 0.870 g/cm³ according to ASTM D792, heat of fusion of 46.1 J/g, and a Brookfield viscosity of 8200 cP at 177° C. according to ASTM D 1084. AFFINITY™ GA 1950 has a density of 0.874 g/cm³ according to ASTM D792, heat of fusion of 53.4 J/g, and a Brookfield viscosity of 17,000 cP at 177° C. according to ASTM D 1084. ENGAGE™ 8200 has a density of 0.87 g/cm³ according to ASTM D792 and a melt index of 5 g/10 min at 190° C. These olefin elastomers are compatible with the propylene copolymers useful in the hot melt adhesive compositions and improve physical properties such as low temperature adhesive performance without sacrificing effective set time.

Any conventional polymerization synthesis processes may prepare the polyolefin copolymers. Preferably, one or more catalysts, which are typically metallocene catalysts or Zeigler-Natta, catalysts, are used for polymerization of an olefin monomer or monomer mixture. Polymerization methods include high pressure, slurry, gas, bulk, suspension, supercritical, or solution phase, or a combination thereof, preferably using a single-site metallocene catalyst system. The catalysts can be in the form of a homogeneous solution, supported, or a combination thereof. Polymerization may be carried out by a continuous, a semi-continuous or batch process and may include use of chain transfer agents, scavengers, or other such additives as deemed applicable. By continuous is meant a system that operates (or is intended to operate) without interruption or cessation. For example a continuous process to produce a polymer would be one where the reactants are continually introduced into one or more reactors and polymer product is continually withdrawn. In one embodiment, the propene copolymer described herein is produced in a single or multiple polymerization zones using a single polymerization catalyst. The heterophase polymers are typically made using multiple metallocene catalyst blends that obtain desired heterophase structure.

In some embodiments, the adhesive may comprise an amorphous polyolefin copolymer composition comprising more than 40 mole % 1-butene and a second amorphous polymer comprising at least one butene monomer, wherein the polymer is compatible with the polyolefin. In some embodiments, the adhesive may consist essentially of an amorphous polyolefin copolymer composition comprising more than 40 mole % 1-butene and a compatible second amorphous polymer comprising at least one butene monomer. The second polymer compatible with the polyolefin may have a molecular weight (MWₙ) of at least 1000. Such compatibility arises from a liquid amorphous material comprising at least one butene monomer (1-butene, cis and trans-2-butene, and isobutylene) isomer. Unlike conventional plasticizing oils such as white oils having a conventional hydrocarbon character, useful materials are sufficiently compatible and as a result improve add-on processability characteristics, reduce viscosity, and maintain adhesive bond while improving cohesive properties. The term “compatible or compatibility” of a blend of polymers, as the term is used in this disclosure, means that (1) the materials blend into a uniform hot melt and (2) the cohesive strength of a mixture (70/30 to 50/50) by weight of the amorphous 1-butene polymer and the second amorphous polymer is maintained for construction purposes. Preferred materials comprise a compatible extender, diluents, and viscosity modifier such as a polyisobutylene polymer. The polymer can comprise major proportion of isobutylene units or can be represented as:

[—C(CH₃)₂—CH₂-]ₙ;

wherein n=15 to 75. Preferred materials such as a polyisobutylene are viscous liquids with molecular weight of about 200-20,000, about 200-5,000 or about 500-3,000. The preferred liquid materials have a Saybolt Universal seconds (SUS) viscosity at 100° C. of about 100 to 20,000. The characteristic features of polyisobutylene are low gas permeability and high resistance to the action of acids, alkalis, and solutions of salts, as well as high dielectric indexes. They degrade gradually under the action of sunlight and ultraviolet rays (the addition of carbon black slows this process). In industry, polyisobutylene is produced by ionic (AlCl₃ catalyzed) polymerization of the monomer at temperatures from −80° to −100° C.; they are processed using the ordinary equipment of the rubber industry. Polyisobutylene combines easily with natural or synthetic rubbers, polyethylene, polyvinyl chloride, and phenol-formaldehyde resins.

Any of the compositions disclosed herein can also comprise a plasticizer or plasticizing oil or extender oil that may reduce viscosity or improve tack properties in the adhesive. Any plasticizer known to a person of ordinary skill in the art may be used in the adhesion compositions disclosed herein. Nonlimiting examples of plasticizers include olefin oligomers, low molecular weight polyolefin such as liquid polybutene, low molecular weight non-aromatic polymers (e.g. REGALREZ 101 from Eastman Chemical Company), phthalates, mineral oils such as naphthenic, paraffinic, or hydrogenated (white) oils (e.g. Kaydol oil or ParaLux oils (Chevron U.S.A. Inc.)), vegetable and animal oil and their derivatives, petroleum derived oils, and combinations thereof. Low molecular weight polyolefin may include those with Mw as low as 100, in particular, those in the range of from about 100 to 3000, in the range of from about 250 to about 2000 and in the range of from about 300 to about 1000.

In some embodiments, the plasticizers include polypropylene, polybutene, hydrogenated polyisoprene, hydrogenated polybutadiene, polypiperylene, copolymers of piperylene and isoprene, and the like, having average molecular weights between about 350 and about 10,000. In other embodiments, the plasticizers include glyceryl esters of the usual fatty acids and polymerization products thereof a polymer of isobutylene.

As noted above, embodiments of preferred compositions are made with substantially less than 40 wt. %, less than 20 wt. % or are substantially free of an effective amount of a conventional tackifier material that can add any aspect of open time, substrate wetting or tack to the adhesive material. Avoiding the use of a tackifier reduces adhesive density, adhesive and product costs, and frees formulators from the use of materials in short supply. Further, tackifier can impart undesirable odor in disposable articles and can also act as carriers of low molecular weight plasticizers (like process oils that are used in SBC based adhesives) that can weaken the polyethylene film materials used in baby diapers. For example, back sheet integrity is becoming more important due to the downsizing of the polyethylene film thickness used in these articles. By the term “conventional tackifier resins”, those resins commonly available in the adhesive art and industry that are used in typical hot melt adhesives. Examples of conventional tackifing resins included in this range include an aliphatic hydrocarbon resins, aromatic modified aliphatic hydrocarbon resins, hydrogenated poly-cyclopentadiene resins, poly-cyclopentadiene resins, gum rosins, gum rosin esters, wood rosins, wood rosin esters, tall oil rosins, tall oil rosin esters, poly-terpene, aromatic modified poly-terpene, terpene-phenolic, aromatic modified hydrogenated poly-cyclopentadiene resins, hydrogenated aliphatic resins, hydrogenated aliphatic aromatic resins, hydrogenated terpene and modified terpene and hydrogenated rosin esters. Often in conventional formulations such resins are used in amounts that range from about 5 to about 65 wt. %, often about 20 to 30 wt. %.

In further embodiments, the compositions disclosed herein optionally can comprise an antioxidant or a stabilizer. Any antioxidant known to a person of ordinary skill in the art may be used in the adhesion composition disclosed herein. Non-limiting examples of suitable antioxidants include amine-based antioxidants such as alkyl diphenyl amines, phenyl-naphthylamine, alkyl or aralkyl substituted phenyl-naphthylamine, alkylated p-phenylene diamines, tetramethyl-diaminodiphenylamine and the like; and hindered phenol compounds such as 2,6-di-t-butyl-4-methylphenol; 1,3,5-trimethyl-2,4,6-tris(3′,5′-di-t-butyl-4′-hydroxybenzyl)benzene; tetra kis[(methylene(3,5-di-t-butyl-4-hydroxyhydrocinnamate)]methane (e.g., IRGANOX™1010, from Ciba Geigy, New York); octadecyl-3,5-di-t-butyl-4-hydroxycinnamate (e.g., IRGANOX™ 1076, commercially available from Ciba Geigy) and combinations thereof. Where used, the amount of the antioxidant in the composition can be from about greater than 0 to about 1 wt. %, from about 0.05 to about 0.75 wt. %, or from about 0.1 to about 0.5 wt. % of the total weight of the composition.

In further embodiments, the compositions disclosed herein optionally can comprise an UV stabilizer that may prevent or reduce the degradation of the composition by radiation. Any UV stabilizer known to a person of ordinary skill in the art may be used in the adhesion composition disclosed herein. Non-limiting examples of suitable UV stabilizers include benzophenones, benzotriazoles, aryl esters, oxanilides, acrylic esters, formamidine carbon black, hindered amines, nickel quenchers, hindered amines, phenolic antioxidants, metallic salts, zinc compounds and combinations thereof. Where used, the amount of the UV stabilizer in the composition can be from about greater than 0 to about 1 wt. %, from about 0.05 to about 0.75 wt. %, or from about 0.1 to about 0.5 wt. % of the total weight of the composition.

In further embodiments, the compositions disclosed herein optionally can comprise a brightener, colorant or pigment. Any colorant or pigment known to a person of ordinary skill in the art may be used in the adhesion composition disclosed herein. Non-limiting examples of suitable brighteners, colorants or pigments include fluorescent materials and pigments such as triazine-stilbene, coumarin, imidazole, diazole, titanium dioxide and carbon black, phthalocyanine pigments, and other organic pigments such as IRGAZINB, CROMOPHTALB, MONASTRALB, CINQUASIAB, IRGALITEB, ORASOLB, all of which are available from Ciba Specialty Chemicals, Tarrytown, N.Y. Where used, the amount of the brightener, colorant or pigment in the composition can be from about greater than 0 to about 10 wt %, from about 0.01 to about 5 wt %, or from about 0.1 to about 2 wt % of the total weight of the composition.

The compositions disclosed herein may also optionally comprise a fragrance such as a perfume or other odorant. Such fragrances may be retained by a liner or contained in release agents such as microcapsules that may, for example, release fragrance upon removal of a release liner from or compression on the composition.

In further embodiments, the compositions disclosed herein optionally can comprise filler. Any filler known to a person of ordinary skill in the art may be used in the adhesion composition disclosed herein. Non-limiting examples of suitable fillers include sand, talc, dolomite, calcium carbonate, clay, silica, mica, wollastonite, feldspar, aluminum silicate, alumina, hydrated alumina, glass bead, glass microsphere, ceramic microsphere, thermoplastic microsphere, barite, wood flour, and combinations thereof. Where used, the amount of the filler in the composition can be from about greater than 0 to about 60 wt. %, from about 1 to about 50 wt. %, or from about 5 to about 40 wt. %

TABLE 1
Exemplary and Useful
Substantially Tackifier Free Adhesive Compositions

| Component | Embodiment | Wt. % | Wt. % | Wt. % |
|---|---|---|---|---|
| Amorphous | REXTAC E65 | 90-10 | 20-80 | 70-40 |
| polymer | | | | |
| Heterophase | Vistamaxx | 10-90 | 80-20 | 40-70 |
| polymer | | | | |
| Plasticizer | Polyisobutylene | 0-40 | 5-35 | 5-30 |
| Additive | Antioxidant/stabilizer | 0-20 | 1-20 | 1-15 |

TABLE 2
Exemplary Tackifier-Free Adhesive Compositions

| Component | Embodiment | Wt. % | Wt. % | Wt. % |
|---|---|---|---|---|
| Amorphous | REXTAC E63 or | 90-10 | 30-85 | 75-40 |
| polymer | E65 or blends | | | |
| | (Sustic | | | |
| | technology) | | | |
| Second | Polyisobutylene | 0-50 | 5-45 | 5-40 |
| amorphous | | | | |
| polymer | | | | |
| Additive | Extender/diluent | 0-30 | 0.1-20 | 0.1-10 |
| Additive | Brightener | 0.001-0.3 | 0.001-0.1 | 0.001-0.05 |
| Additive | Antioxidant/ | 0-20 | 1-20 | 1-15 |
| | stabilizer | | | |

One substantial advantage in the claimed adhesives relates to a density of the adhesive formulations. Conventional tackifier is at a density that often ranges from about 1.07-1.09 g-cm⁻³. Conventional formulated adhesives containing a conventional tackifier in amounts of about 40 to 60 wt. %, have a density greater than 0.9 g-cm⁻³ or more. The formulated adhesives of the invention, substantially free of tackifier, have densities less than 0.9 g-cm⁻³, often in the range about 0.85-0.89 g-cm⁻³ often 0.86-0.87 g-cm⁻³. Not only are these adhesives free of the problems arising from tackifier materials, but the use of the claimed adhesives, and a lower density, permits the use of a reduced amount when measured by weight, resulting in cost savings.

Another aspect is methods of manufacture employing the hot melt adhesive compositions. The method involves application of the molten compositions to a substrate, followed by contact of the adhesive composition with a second substrate within 0.1 second to 5 seconds after application of the adhesive composition to the first substrate, wherein the contacting results in an adhesive bond between the substrates.

The hot melt adhesive compositions have melt rheology and thermal stability suitable for use with conventional hot melt adhesive application equipment. The blended components of the hot melt adhesive compositions have low melt viscosity at the application temperature, thereby facilitating flow of the compositions through a coating apparatus, e.g., coating die or nozzle, without resorting to the inclusion of solvents or extender oil into the composition. Melt viscosities of the hot melt adhesive compositions are between 1500 cP and 3500 cP or about 2000 cP to 3000 cP in mille Pascal-seconds or centipoise (cP) using a Brookfield thermosel RVT viscometer using a rotor number 27 at 176.66° C. (50 rpm, 350° F.). The hot melt adhesive compositions have a softening point (ASTM D 3461-97 Standard Test Method for Mettler Softening Point Method) of about 80° C. to 140° C., in some embodiments about 115° C. to 130° C. For certain applications, the hot melt adhesive compositions have effective set times of about 5 seconds or less, for example about 0.1 second to 5 seconds, in embodiments about 0.1 second to 3 seconds, and in some embodiments about 0.2 second to 1 second. The effective set time of the hot melt adhesives are unexpectedly short, particularly given that the open time remains in the acceptable range.

The adhesives described herein may be used to bond any of the substrates of the belted structures described herein. Specific examples include, but are not limited to, the belt structure to the central chassis; the belt structure outer nonwoven to the belt structure inner nonwoven; the elastic strands to the belt structure nonwovens; and the respective front and rear left side edges and the front and rear right side edges.

The adhesive is typically applied in an amount of about 1 to about 100 or about 4 to about 90 or about 7 to about 70 grams per square meter (g/m²) of resulting bonded material. The material may be applied in an amount of about 0.1 to about 20 or about 0.2 to about 10 or about 0.3 to about 15 grams per square meter (g/m²) of resulting bonded material. The adhesive material can be used at an add-on rate of 0.5 to 2 g/m², 0.6 to 1.7 g/m² or 0.7 to 1.5 g/m², for absorbent articles.

### EXAMPLES

A number of hot melt adhesive compositions were prepared by blending first amorphous copolymer, second heterophase copolymer, polymer plasticizer/diluent and antioxidant under mixing conditions at elevated temperatures to form a fully homogenized fluid melt. Mixing temperatures varied from about 135 to about 200° C. preferably about 150 to about 175° C. A WiseStir® mixer was used to ensure full homogenization of components into a final adhesive composition.

### Examples 1-8

Hot melt adhesive compositions were formulated by melt blending as described below, wherein specific components and amounts of the components are shown in the following table 3.

TABLE 3
Exemplary Adhesive Formulations

| | | Ex. 1 | Ex. 2 | Ex. 3 | Ex. 4 | Ex. 5 | Ex. 6 | Ex. 7 | Ex. 8 |
|---|---|---|---|---|---|---|---|---|---|
| Source | Component | wt. % | wt. % | wt. % | wt. % | wt. % | wt. % | wt. % | wt. % |
| ExxonMobil | Vistamaxx | 20 | 35 | 35 | 35 | 15 | 15 | 15 | 10 |
| Chemical, | 8816 | | | | | | | | |
| Houston, TX | | | | | | | | | |
| Huntsman | Rextac E-65 | 59.5 | 60 | 55 | 50 | 64.5 | 59.5 | 59.5 | 59.5 |
| Chemicals | | | | | | | | | |
| Ineos | Indapol H- | 20 | 4.5 | 9.5 | 14.5 | 20 | 24.99 | 0 | 0 |
| Chemicals | 300 (Polyiso- | | | | | | | | |
| | butylene) | | | | | | | | |
| Ineos | Indapol H- | 0 | 0 | 0 | 0 | 0 | 0.5 | 0.5 | 0.5 |
| Chemicals | 1900 | | | | | | | | |
| | (Polyiso- | | | | | | | | |
| | butylene) | | | | | | | | |
| Ciba Geigy | Irganox 1010 | 0.5 | 0.5 | 0.5 | 0.5 | 0.5 | 0.5 | 0.5 | 0.5 |
| Ltd., Basel, | (Hindered | | | | | | | | |
| Switzerland | Phenol) | | | | | | | | |
| Mayzo Inc. | Benetex OB | 0 | 0 | 0 | 0 | 0 | 0.01 | 0.01 | 0.01 |
| Fluorescent | | | | | | | | | |
| Optical | | | | | | | | | |
| Brightener | | | | | | | | | |

TABLE 4
Exemplary Adhesive Viscosity Data

| Brookfield | | | | | | | | |
|---|---|---|---|---|---|---|---|---|
| Viscosity @ | Ex. 1 | Ex. 2 | Ex. 3 | Ex. 4 | Ex. 5 | Ex. 6 | Ex. 7 | Ex. 8 |
| 121.1° C. (250° F.) | 26200 | | | | 29750 | 16600 | | 39000 |
| 135° C. (275° F.) | 7710 | 12125 | 9725 | 7500 | 8425 | 7100 | 9100 | 8750 |
| 148.9° C. (300° F.) | 4675 | 6350 | 5325 | 4525 | 5150 | 4200 | 5325 | 5375 |
| 162.8° C. (325° F.) | 3075 | 4190 | 3500 | 2980 | 3475 | 2800 | 3550 | 3375 |
| 176.7° C. (350° F.) | 2220 | 2945 | 2450 | 2080 | 2315 | 1920 | 2385 | 2275 |
| Mettler Softening | 121 | 125 | 125 | 124 | 120 | 118 | 118 | 115 |
| Point (° C.) | | | | | | | | |
| Density g/cm³ | 0.86- | 0.86- | 0.86- | 0.86- | 0.86- | 0.86- | 0.86- | 0.86- |
| ASTM 792 | 0.87 | 0.87 | 0.87 | 0.87 | 0.87 | 0.87 | 0.87 | 0.87 |

These data indicates that the materials will provide excellent bonding in disposable absorbent articles. Note viscosity relates to the resistance to flow of the material under certain conditions. This distinctive property determines the flowability, degree of wetting, and penetration of the substrate by the molten polymer. It provides an indication of its processability and utility as a hot melt adhesive material. Melt viscosity is generally directly related to a polymer molecular weight and is reported in Millipascal-second's or centipoise (cP) using a Brookfield thermosel RVT viscometer using a rotor number 27 at the stated temperature.

Mettler softening point in degrees Centigrade or degrees Fahrenheit is typically measured using ASTM D3104. The amorphous nature of the poly olefin materials results in a melting point, which is not sharp or definite. Rather as the temperature increases, amorphous polymers gradually change from a solid to a soft and then to a liquid material. No clearly defined glass transition or melting temperature is often noted. This temperature testament that generally measures the precise temperature at which a disc of polymer sample, heated at a rate of 2° C. per minute or 10° per minute becomes soft enough to allow the test object, a steel ball (grams) drops through the sample. The softening point of a polymer reported in degrees Centigrade or degrees Fahrenheit is important because it typically indicates the polymer's heat resistance, useful application temperatures and solidification points.

### Examples 9-11

A number of hot melt adhesive compositions were prepared by blending first amorphous copolymer, second compatible copolymer and antioxidant under mixing conditions at elevated temperatures to form a fully homogenized melt. Mixing temperatures varied from about 135 to about 200° C. preferably about 150 to about 175° C. as needed to obtain uniformity. A traditional heated stirred blade (WiseStir®) mixer was used to ensure full homogenization in a heated container into a final adhesive composition.

### Examples 9-11

Hot melt adhesive compositions were formulated by melt blending, as described below, wherein specific components and amounts of the components are shown in the following table 5.

TABLE 5
Experimental Preparations

| | Ex. 9 | Ex. 10 | Ex. 11 |
|---|---|---|---|
| Component | (wt. %) | (wt. %) | (wt. %) |
| Rextac E-65 (1-butene copolymer) | 44.5 | 54.5 | |
| Rextac E-63 (1-butene copolymer) | 30 | 20 | |
| Rextac 2830 (1-butene copolymer) | | | 70 |
| Indapol H-1900 | 24.99 | 24.99 | 29.49 |
| Polyisobutylene (MW 2500) | | | |
| Irganox 1010 (stabilizer) | 0.5 | 0.5 | 0.5 |
| B en otex OB | 0.01 | 0.01 | 0.01 |
| (Optical brightener) | | | |
| Brookfield DV-II + pro | | | |
| Viscosity (cP) Rotation 10 rpm | | | |
| Sprindle # SC4-27 | | | |
| 250° F. | 31000 | 23825 | 18200 |
| 275° F. | 13650 | 13175 | 10250 |
| 300° F. | 6265 | 6875 | 6050 |
| 325° F. | 4090 | 4460 | 3850 |
| 350° F. | 3245 | 3060 | 2595 |
| Mettler Softening Point (° C.) | 116 | 115 | 91 |
| Density (g/cm³) | 0.87 | 0.87 | 0.87 |

### Comparative Example 1

Hot melt adhesive compositions are formulated by melt blending, as described below, wherein specific components and amounts of the components are shown in the following table 6. Comparative examples 1 and 2 each form a non-uniform composition that has insufficient cohesive/adhesive strength to be usefully measured.

| | | | |
|---|---|---|---|
| Component | CEx. 1 (wt. %) | CEx. | 2 (wt. %) |

| | | |
|---|---|---|
| APAO | | 75 |
| Rextac E-63 | 75 | |
| (1-butene copolymer) | | |
| Polyisobutylene | | 25 |
| White Oil | 25 | |
| Irganox 1010 (Stabilizer) | 0 | 0 |
| Benotex OB (Optical | 0 | 0 |
| brightener) | | |

TABLE 7
Test Results

| | Add-on | Add-on | | | | | | | | |
|---|---|---|---|---|---|---|---|---|---|---|
| | method - | (g/m²) | | | Air | | | | | |
| | Nordsen ®) | over | | | Press. | Web Speed | | Peak | Ave. | Peel |
| | Hot Melt | 120 mm | Temp | Gap | (psi/ | (inch-sec⁻¹/ | | Peel | Peel | force |
| Run | applic. | width | (° F./° C.) | (mm) | Pascal) | m-sec⁻¹) | Ex. | (g/in) | (g/in) | (N/cm) |
| 1 | Slot/true | 0.75 | 320/160 | | | 2000/50.8 | Ex. 10 | 190 | 93 | 0.37 |
| | coat die | | | | | | | | | |
| 2 | Slot/true | 1 | 310/154.4 | | | 2000/50.8 | Ex. 10 | 202 | 110 | 0.43 |
| | coat die | | | | | | | | | |
| 3 | Slot/true | 1 | 320/160 | | | 2000/50.8 | Ex. 10 | 217 | 134 | 0.53 |
| | coat die | | | | | | | | | |
| 4 | Slot/true | 1 | 330/165.6 | | | 2000/50.8 | Ex. 10 | 212 | 131 | 0.52 |
| | coat die | | | | | | | | | |
| 5 | Slot/true | 1 | 315/157.2 | | | 2000/50.8 | Ex. 10 | 205 | 110 | 0.43 |
| | coat die | | | | | | | | | |
| 6 | Slot/true | 0.5 | 320/160 | | | 2000/50.8 | Ex. 10 | 111 | 58 | 0.23 |
| | coat die | | | | | | | | | |
| 7 | Slot/true | 0.75 | 320/160 | | | 2000/50.8 | Ex. 10 | 161 | 95 | 0.37 |
| | coat die | | | | | | | | | |
| 8 | Slot/true | 0.5 | 320/160 | | | 2000/50.8 | Ex. 9 | 126 | 70 | 0.28 |
| | coat die | | | | | | | | | |
| 9 | Slot/true | 0.75 | 320/160 | | | 2000/50.8 | Ex. 9 | 181 | 100 | 0.39 |
| | coat die | | | | | | | | | |
| 10 | Slot/true | 0.5 | 320/160 | | | 2000/50.8 | Ex. 11 | 117 | 62 | 0.24 |
| | coat die | | | | | | | | | |
| 11 | Slot/true | 0.75 | 320/160 | | | 2000/50.8 | Ex. 11 | 152 | 93 | 0.37 |
| | coat die | | | | | | | | | |
| 12 | Slot/true | 1 | 320/160 | | | 2000/50.8 | Ex. 11 | 192 | 123 | 0.48 |
| | coat die | | | | | | | | | |
| 13 | Signature | 1 | 360/182.2 | 20 | 40/0.276 | 2000/50.8 | Ex. 10 | 154 | 92 | 0.36 |
| 14 | Signature | 1 | 360/182.2 | 20 | 45/0.310 | 2000/50.8 | Ex. 10 | 164 | 96 | 0.38 |
| 15 | Signature | 1 | 360/182.2 | 25 | 45/0.310 | 2000/50.8 | Ex. 10 | 189 | 102 | 0.4 |
| 16 | Signature | 1.25 | 360/182.2 | 25 | 45/0.310 | 2000/50.8 | Ex. 10 | 201 | 123 | 0.48 |
| 17 | Signature | 1.25 | 360/182.2 | 25 | 45/0.310 | 2000/50.8 | Ex. 11 | 187 | 116 | 0.46 |
| 18 | Signature | 1 | 360/182.2 | 25 | 45/0.310 | 2000/50.8 | Ex. 11 | 158 | 88 | 0.35 |
| 19 | Signature | 1 | 360/182.2 | 25 | 45/0.310 | 2000/50.8 | Ex. 9 | 197 | 122 | 0.48 |
| 20 | Signature | 1.25 | 360/182.2 | 25 | 45/0.310 | 2000/50.8 | Ex. 9 | 232 | 138 | 0.54 |

All tests show adhesion and good bonding. The data from runs 2, 3, 4, 5, 9, 12, 15, 16, 17, 19, and 20 show values that all exceeded requirements for a successful construction adhesive for absorbent articles.

These data indicates that the materials will provide excellent bonding in disposable absorbent articles. Note viscosity relates to the resistance to flow of the material under certain conditions. This distinctive property determines the flowability, degree of wetting, and penetration of the substrate by the molten polymer. It provides an indication of its processability and utility as a hot melt adhesive material.

Melt viscosity is generally directly related to a polymer molecular weight and is reported in millipascal-second (mP·s) or centipoise (cP) using a Brookfield DV-II+Pro (Rotation 10 rpm—Spindle # SC4-27) at the stated temperature.

Mettler softening point in degrees Centigrade or degrees Fahrenheit is typically measured using ASTM D3104. The amorphous nature of the polyolefin materials results in a melting point, which is not sharp or definite. Rather as the temperature increases, amorphous polymers gradually change from a solid to a soft and then to a liquid material. No clearly defined glass transition or melting temperature is often noted. This temperature testament that generally measures the precise temperature at which a disc of polymer sample, heated at a rate of 2° C. per minute or 10° F. per minute becomes soft enough to allow the test object, a steel ball (grams) drops through the sample. The softening point of a polymer reported in degrees Centigrade or degrees Fahrenheit is important because it typically indicates the polymer's heat resistance, useful application temperatures and solidification points.

Peel test values were obtained by forming a laminate from a SMS non-woven (11.6 g/m²) micro-porous polyethylene film (0.5 mil/0.127 micron) using lamination conditions as shown in Table 4. The laminate is cut into 1 inch/25.4 mm wide strips in the cross machine direction. Peel force was measured by separating the laminate at room temperature using a TMax pull tester at a rate of 20 in/sec (50.8 cm/sec) with the peek force averaged over a 15 period.

The claims may suitably comprise, consist of, or consist essentially of, or be substantially free of any of the disclosed or recited elements. The invention illustratively disclosed herein can also be suitably practiced in the absence of any element which is not specifically disclosed herein.

The dimensions and values disclosed herein are not to be understood as being strictly limited to the exact numeral values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a front region, a rear region, and a crotch region disposed therebetween; and comprising:
a liquid permeable topsheet; a backsheet, and an absorbent core disposed between the topsheet and the backsheet;
a central chassis occupying the crotch region;
a belt structure disposed about the central chassis, the belt structure overlaying the backsheet to the outside thereof in the front and rear regions, the belt structure overlapping and extending laterally and longitudinally outward from the chassis;
wherein the belt structure comprises an outer nonwoven and an inner nonwoven and elastic strands therebetween;
the belt structure further having a front belt portion having a front waist edge, and front left and right side edges; and a rear belt portion having a rear waist edge and rear left and right side edges;
wherein respective front and rear left side edges and the respective front and rear right side edges are joined, forming a waist opening and left and right leg openings;
wherein an adhesive bonds at least one of the group consisting of the belt structure to the central chassis; the belt structure outer nonwoven to the belt structure inner nonwoven; the elastic strands to the belt structure nonwovens; and the respective front and rear left side edges and the front and rear right side edges;
wherein the adhesive comprises:
an amorphous polyolefin composition; and
a heterophase polyolefin composition comprising amorphous character and crystalline blocks;
wherein the adhesive has a density of less than 0.9 g/cm³; and
wherein the adhesive is substantially tackifier-free.

2. The absorbent article of claim 1, wherein the amorphous polyolefin has less than 5 wt. % crystallinity and the heterophase polyolefin comprises at least about 5 wt. % crystallinity in at least one sequence or block; and wherein the amorphous polyolefin provides adhesion and the heterophase polyolefin provides cohesive strength.

3. The absorbent article of claim 1, wherein the amorphous polyolefin comprises greater than 40 wt. % butene and less than 50 wt. % of one or more alpha olefin C₂ or C₄₋₂₀ monomers.

4. The absorbent article of claim 1, wherein the heterophase polyolefin comprises greater than 40 wt. % of propene and less than 60 wt. % of one or more alpha olefin C₂ or C₂₋₂₀ monomers and comprises polymer blocks or sequences that have a crystallinity of greater than 10%.

5. The absorbent article of claim 1, wherein the adhesive further comprises a plasticizer.

6. The absorbent article of claim 5, wherein the plasticizer comprises polyisobutylene with a molecular weight of about 500 to about 2000.

7. The absorbent article of claim 1, comprising a first leg elastic disposed adjacent to the left leg opening and a second leg elastic disposed adjacent to the right leg opening, wherein the first and second leg elastics do not overlap the central chassis.

8. The absorbent article of claim 7, wherein the adhesive joins the first and second leg elastics to the belt structure.

9. An absorbent article having a front region, a rear region, and a crotch region disposed therebetween; and comprising:
a liquid permeable topsheet; a backsheet, and an absorbent core disposed between the topsheet and the backsheet;
a central chassis occupying the crotch region;
a belt structure disposed about the central chassis, the belt structure overlaying the backsheet to the outside thereof in the front and rear regions, the belt structure overlapping and extending laterally and longitudinally outward from the chassis;
wherein the belt structure comprises an outer nonwoven and an inner nonwoven and elastic strands therebetween;
the belt structure further having a front belt portion having a front waist edge, and front left and right side edges; and a rear belt portion having a rear waist edge and rear left and right side edges;
wherein respective front and rear left side edges and the respective front and rear right side edges are joined, forming a waist opening and left and right leg openings;
wherein an adhesive bonds at least one of the group consisting of the belt structure to the central chassis; the belt structure outer nonwoven to the belt structure inner nonwoven; the elastic strands to the belt structure nonwovens; and the respective front and rear left side edges and the front and rear right side edges;
wherein the substantially tackifier-free adhesive consists essentially of:
an amorphous polyolefin composition comprising more than 40% 1-butene; and
a second amorphous polymer comprising at least one butene monomer, the polymer having a molecular weight (MWₙ) of at least 1000 wherein the polymer is compatible with the polyolefin;
wherein the adhesive has a density of less than 0.9 g/cm³; and
wherein the adhesive is substantially tackifier-free.

10. The absorbent article of claim 9, wherein the second amorphous polymer comprises a viscous liquid with molecular weight of about 200-20,000 and a Saybolt Universal seconds (SUS) viscosity at 100° C. of about 100 to 20,000.

11. The absorbent article of claim 9, wherein the amorphous polyolefin polymer comprises less than 50 wt. % of one or more alpha olefin C₂ or C₄₋₂₀ monomers.

12. The absorbent article of claim 9, wherein the second amorphous polymer comprises a polyisobutylene with a molecular weight of 1500 to 6000.

13. The absorbent article of claim 9, wherein the adhesive comprises about 50 to 90 wt. % of the amorphous polymer and about 10 to 50 wt. % of the polyisobutylene.

14. The absorbent article of claim 9, comprising a first leg elastic disposed adjacent to the left leg opening and a second leg elastic disposed adjacent to the right leg opening, wherein the first and second leg elastics do not overlap the central chassis.

15. The absorbent article of claim 14, wherein the adhesive joins the first and second leg elastics to the belt structure.
